(19) United States
(12) Patent Application Publication
(10) Pub. No.: US 2025/0215502 A1
(43) Pub. Date: Jul. 3, 2025

(54) DNA METHYLATION BIOMARKERS FOR DETECTION OF HIGH-GRADE DYSPLASIA AND ESOPHAGEAL OR JUNCTIONAL ADENOCARCINOMA

(71) Applicant: Fred Hutchinson Cancer Center, Seattle, WA (US)

(72) Inventors: William M. Grady, Seattle, WA (US); Ming Yu, Bellevue, WA (US); Amber Willbanks, Seattle, WA (US)

(73) Assignee: Fred Hutchinson Cancer Center, Seattle, WA (US)

(21) Appl. No.: 18/844,826

(22) PCT Filed: Mar. 8, 2023
(86) PCT No.: PCT/US2023/063976

§ 371 Date: Sep. 6, 2024

Related U.S. Application Data
(60) Provisional application No. 63/317,906, filed on Mar. 8, 2022.

(51) Int. Cl. C12Q 1/6886

(52) U.S. Cl. CPC C12Q 1/6886; C12Q 2600/112; C12Q 2600/154

(57) ABSTRACT

Methods and kits to distinguish normal or Barrett's esophageal samples from high grade dysplasia (HGD), esophageal adenocarcinoma (EAC), or junctional adenocarcinoma (JCA) samples are described. The methods and kits utilize the methylation status of epigenetic markers, such as cg6522, POU3F1, YPEL3, and/or MAFB.

### CROSS REFERENCE TO RELATED APPLICATIONS

This application is a U.S. National Phase Application Based on International Patent Application No. PCT/US2023/063976, which claims priority to U.S. Provisional Patent Application No. 63/317,906 filed Mar. 8, 2022, both of which are[[is]] incorporated herein by reference in its entirety as if fully set forth herein.

### STATEMENT OF GOVERNMENT SUPPORT

This invention was made with government support under CA015704, CA182940, CA233042, CA152756, and CA220004 awarded by the National Institutes of Health. The government has certain rights in the invention.

### REFERENCE TO SEQUENCE LISTING

The Sequence Listing associated with this application is provided in text format in lieu of a paper copy, and is hereby incorporated by reference into the specification. The name of the text file containing the Sequence Listing is 3AL7938.XML. The text file is 24,567 bytes, was created on Sep. 5, 2024, and is being submitted electronically via Patent Center.

### FIELD OF THE DISCLOSURE

The present disclosure provides minimally-invasive methods and kits to distinguish normal or Barrett's esophageal samples from high grade dysplasia (HGD), esophageal adenocarcinoma (EAC), or junction adenocarcinoma (JCA) samples using the methylation status of epigenetic markers. The epigenetic markers include cg6522, POU3F1, YPEL3, and MAFB, or utilize a logistic regression model utilizing the two-markers cg6522 and POU3F1. Following detection of HGD, EAC, or JCA, subjects can be referred for follow-up treatment.

### BACKGROUND OF THE DISCLOSURE

Esophageal adenocarcinoma (EAC) incidence is rapidly increasing in the US for both known and unknown reasons and affects 20,000 people each year. It has a poor prognosis with <20% of patients surviving 5 years. Survival rates dramatically improve if EAC is detected at an early stage, when it can be cured by surgical or endoscopic resection. Importantly, virtually all EAC is believed to arise from a precancerous condition called Barrett's esophagus (BE), which can evolve into EAC over time through a BE to high grade dysplasia (HGD) to EAC progression sequence. An important recent development in the treatment of HGD and early EAC are radiofrequency ablation (RFA) and intramucosal resection (EMR), which are endoscopic therapies that can cure these lesions. HGD and early stage EAC can be removed with RFA and EMR with low morbidity and mortality, substantially reducing the risk for EAC therapy related death. Given this low morbidity and low mortality treatment option, assays that can detect HGD, early stage EAC, and/or the rare but lethal junctional adenocarcinoma (JCA) are of high value and have the potential to reduce EAC related death and preserve the quality of life of BE patients.

### SUMMARY OF THE DISCLOSURE

The present disclosure provides minimally-invasive methods and kits to distinguish normal or Barrett's esophageal samples from high grade dysplasia (HGD), esophageal adenocarcinoma (EAC), or junctional adenocarcinoma (JCA) using the methylation status of epigenetic markers. The epigenetic markers include cg6522, POU3F1, YPEL3, and MAFB. A two-marker panel of cg6522 and POU3F1 can also be used. Following detection of HGD, EAC, or JCA, subjects can be referred for follow-up treatment.

### BRIEF DESCRIPTION OF THE FIGURES

Some of the drawings submitted herein may be better understood in color. Applicant considers the color versions of the drawings as part of the original submission and reserves the right to present color images of the drawings in later proceedings.

FIG. 1. Workflow for identification of DNA-methylation biomarkers for HGD/EAC early detection.

FIGS. 2A-2D. Dot-plot graphs of individual markers (2A) cg6522, (2B) YPEL3, (2C) POU3F1 and (2D) MAFB, in DNA extracted from biopsy samples. Each sample was defined by histologic diagnosis, shown on x-axis, as determined by an experienced pathologist. The y-axis shows the relative methylation percent when compared to the reference gene C-LESS for total DNA, as measured by the methylation-specific droplet digital polymerase chain reaction (MS-ddPCR) assay for each marker. One way ANOVA was used to determine statistical significance between histologic groups based on relative methylation percent. P-values <0.01 when compared to normal tissue are considered significant (*).

FIGS. 3A-3C. Dot-plot graphs of individual markers (3A) cg6522, (3B) YPEL3, and (3C) POU3F1 in DNA extracted from esophageal cytology brushing sample set 1. Each sample was defined by histologic diagnosis, shown on x-axis, as determined by an experienced pathologist. The y-axis shows the relative methylation percent when compared to the reference gene C-LESS for total DNA, as measured by MS-ddPCR assays for each marker. One way ANOVA was used to determine statistical significance between histologic groups based on relative methylation percent. P-values <0.01 when compared to normal tissue are considered significant (*).

FIGS. 4A-4C. Dot-plot graphs of individual markers (4A) cg6522, (4B) YPEL3, and (4C) POU3F1 in DNA extracted from esophageal cytology brushing sample set 2. Each sample was defined by histologic diagnosis, shown on x-axis, as determined by an experienced pathologist. The y-axis shows the relative methylation percent when compared to the reference gene C-LESS for total DNA, as measured by MS-ddPCR assays for each marker. One way ANOVA was used to determine statistical significance between histologic groups based on relative methylation percent. P-values <0.01 when compared to normal tissue are considered significant (*).

FIGS. 5A, 5B. Receiver operating characteristic (ROC) curve of the model −3.7459640+0.1130*cg6522+0.1926*POU3F1 to classify HGD/EAC vs SQ/BE in brushing set 1 (5A) and brushing set 2 (5B) samples with the cutoff p value of 0.45.

### DETAILED DESCRIPTION

In Barrett's esophagus (BE), healthy esophageal epithelium is replaced with metaplastic columnar cells—the result, it is believed, of damage from prolonged exposure of the esophagus to the refluxate of gastroesophageal reflux disease (GERD). The inherent risk of progression from Barrett esophagus to adenocarcinoma of the esophagus has been established. Histologically, this progression involves clear sequential stages from metaplasia alone to low grade dysplasia, then to high grade dysplasia, and finally to adenocarcinoma.

This metaplasia to dysplasia to carcinoma sequence has prompted several national gastroenterology societies to recommend screening for BE in high risk subjects with multiple risk factors followed by endoscopic surveillance (depending on the grade of dysplasia) to detect the development of dysplasia or carcinoma at an early stage (see, Spechler et al., Gastroenterology 201 l; 140:el8-52; Wang et al, Am J Gastroenterol 2008; 103:788-97; Fitzgerald et al, Gut 2014; 63:7-42). Endoscopic treatments of low grade dysplasia (LGD), high grade dyplasia (HGD) and early carcinoma have been developed and shown to be effective in reducing the incidence of carcinoma and improving survival in BE subjects (see, e.g., Prasad et al., Gastroenterology 2007; 132:1226-33; Prasad et al, Gastroenterology 2009; Shaheen et al. N Engl J Med 2009; 360:2277-88; Phoa et al., JAMA 2014; 311:1209-17).

Screening for BE is conventionally performed using sedated endoscopy (sEGD) which reveals the replacement of the normal squamous lining of the esophagus by metaplastic columnar epithelium in subjects with BE. However sedated endoscopy is expensive with both direct and indirect costs and is not suitable for widespread application. It is also associated with potential complications (see, Sami et al, Clinical gastroenterology and hepatology: the official clinical practice journal of the American Gastroenterological Association 2015; 13:623-34). Other techniques such as unsedated transnasal endoscopy (uTNE) have comparable accuracy to sEGD with lower cost, but continue to be poorly regarded as a widely applicable tool by providers (see, Sami et al, The American journal of gastroenterology 2015; 110:148-58; Peery et al, Gastrointestinal endoscopy 2012:75:945-953 e2; Atkinson et al., Gastroenterology & hepatology 2007; 4:426-7). Despite adequate access to the uTNE device, the utilization of uTNE by referring physicians remains limited (see, Atkinson et al, The American journal of gastroenterology 2008; 103:92-7).

Endoscopic detection of dysplasia is conventionally performed using four quadrant random biopsies every 1-2 cm of the BE segment in addition to careful inspection of the BE segment with high resolution white light imaging and advanced imaging techniques. While this has been recommended by GI societies (see, e.g., Spechler et al, Gastroenterology 2011; 140:el 8-52; Wang et al, Am J Gastroenterol 2008; 103:788-97; Fitzgerald et al., Gut 2014; 63:7-42), the compliance with these recommendations amongst practicing gastroenterologists remains poor (see, Abrams et al, Clin Gastroenterol Hepatol 2009). Indeed compliance decreases with increasing BE segment length leading to increasing rates of missed dysplasia. Other challenges with dysplasia detection in BE include the spotty distribution of dysplasia in BE (see, Cameron et al, Am J Gastroenterol 1997; 92:586-91) which leads to sampling error, poor inter-observer agreement amongst pathologists while grading dysplasia, and the relatively poor sensitivity of current surveillance strategies in detecting prevalent dysplasia or carcinoma (see, Sharma et al, Gastroenterology 2004; 127:310-30). The utility of advanced imaging techniques in the community remains unclear with only a third of practicing gastroenterologists reporting use routinely in BE surveillance (see, Singh et al, Gastrointestinal endoscopy 2013; 78:689-95).

Less invasive techniques for esophageal monitoring have been developed. For example, one technique includes a swallowable balloon-based esophageal sampling device for detecting methylated DNAs ((e.g., ESOCHECK™ (Lucid Diagnostics, New York, NY; Moinova et al., Sci Transl Med. 2018; 10(424):eaao5848), RealSeqS analysis of esophageal brushings (Douville et al., Gastroenterology. 2021; 160(6):2043-2054.e2). ESOCHECK™ has been recently approved by the FDA. More particularly, the ESOCHECK™ device is a swallowable sponge on a string developed and studied for use in BE screening (see, Kadri et al, Bmj 2010; 341:c4372). The device includes a polyurethane foam sponge compressed in a gelatin capsule, attached to a string. The capsule is swallowed by the patient. The gelatin shell of the capsule dissolves in the gastric fluid releasing the foam device as a sphere which is then pulled out with the attached string, providing brushing/cytology samples of the proximal stomach and esophagus. Biomarker studies can then be performed on these samples to detect BE. Two large multicenter studies have been performed in the United Kingdom with such a device using trefoil factor 3 (a protein specific to BE epithelium) detected on immunohistochemistry as a BE marker, demonstrating the feasibility, safety and accuracy of this approach (see, Kadri et al., Bmj 2010; 341:c4372; Ross-Innes et al, PLOS medicine 2015; 12:el001780). The sensitivity and specificity of this marker in the detection of BE has been reported to be 73% and 94% for BE segments of >1 cm in circumferential length. Additionally this capsule sponge device has been used safely in a study conducted at Mayo Clinic Rochester in subjects with eosinophilic esophagitis (see, Katzka et al, Clinical gastroenterology and hepatology: the official clinical practice journal of the American Gastroenterological Association 2015; 13:77-83 e2).

The continued and unmet need for non-endoscopic methods for the surveillance of patients with BE led to the currently-disclosed study of whether DNA biomarkers in cytology balloon collected esophageal samples can detect HGD or early EAC.

Biomarker studies have mostly focused on altered gene expression, leading to development of tests for mRNA signatures of tumor progression. Epigenetic alterations in DNA also provides valuable prognostic information. Epigenetics refers to changes in gene expression that are not due to mutations (i.e. changes in the sequence, such as loss or gain of nucleotides, of a gene). Thus, epigenetics is a reversible regulation of gene expression caused by several mechanisms other than mutation.

The most widely studied epigenetic modification is DNA methylation. Other epigenetic changes include changes to the three dimensional structure of DNA, histone protein modification, micro-RNA inhibitory activity, imprinting, X-inactivation, and long-distance chromosomal interaction.

DNA methylation occurs at CpG sites across the genome and regulates gene expression. Cytosine is one of a group of four building blocks (i.e., nucleotides) from which DNA is constructed (i.e. cytosine (C), thiamine (T), adenine (A), and guanosine (G)). The chemical structure of cytosine is in the form of a six-sided hexagon or pyrimidine ring. Cytosine can be paired with guanosine in a linear sequence along the single DNA strand to form 5′-CG-3′, or CpG pairs. “CpG” refers to a cytosine-phosphate-guanosine chemical bond in which the phosphate binds the two nucleotides together. In mammals, in 70-80% of these CpG pairs the cytosine is methylated. (Chatterjee, et al., Biochemica et Biophisica Acta 2012; 1819:763-70).

The term “CpG island” refers to regions in the genome with a high concentration of CG dinucleotide pairs or CpG sites. The length of DNA occupied by the CpG island is usually 300-3000 base pairs. The CpG island can be defined by various criteria including the length of recurrent CG dinucleotide pairs occupying at least 200 base pair (bp) of DNA, a CG content of the segment of at least 50%, and/or that the observed/expected CpG ratio is greater than 60%. There are an estimated 28-30 million CpG sites across the genome.

CpG islands are commonly found in gene promoters. Across mammals, an average of forty percent of gene promoters contain CpG islands (Fatemi, et al., Nucleic Acids Res. 2005; 33:e176). Gene promoters are particularly CG-rich in the human genome, as 70% of promoters in the human genome have high CG content. Although CpG islands are highly associated with gene promoters, CpG islands can also exist in other regions of the genome (such as in gene bodies or in intergenic regions).

In most CpG sites scattered throughout the DNA the cytosine nucleotide is methylated. In contrast, the cytosine is more often unmethylated in CpG sites located in the CpG islands of the promoter regions of genes, supporting a role of methylation status of cytosine in CpG islands in gene transcriptional activity.

Methylation of cytosine refers to the enzymatic addition of a methyl group or single carbon atom to position #5 of the pyrimidine ring of cytosine, which leads to the conversion of cytosine to 5-methyl-cytosine. The methylation of cytosine can be accomplished by a family of enzymes called DNA methyltransferases (DNMTs). The 5-methyl-cytosine, when formed, is prone to mutation or the chemical transformation of the original cytosine to form thymine. Five-methyl-cytosines account for 1% of the nucleotide bases overall in the normal human genome.

As described in the present disclosure, a genome-wide methylation analysis and an exhaustive candidate biomarker search was conducted. A list of candidate methylated DNA biomarkers that showed significantly higher methylation levels in HGD and EAC compared to the normal esophagus and BE samples was identified. More particularly, the present disclosure provides methods and kits to distinguish normal or Barrett's esophageal samples from high grade dysplasia (HGD) or esophageal adenocarcinoma (EAC), or junction adenocarcinoma (JCA) using the methylation status of the epigenetic markers cg6522 (cg41566522), POU3F1 (cg38512601), YPEL3 (cg16348385), MAFB (cg18251612). Certain examples use a two-marker epigenetic panel of cg6522 and POU3F1. Following detection of HGD, EAC, or JCA, subjects can be referred for treatment.

Before bisulfite conversion, the genomic sequences being assessed for methylation status include:

for cg6522:
(SEQ ID NO: 1)
AATGCAGTCGGCCCTGCGCATGTGGTGTCTGAGCAGGGCCCGAGACCTT
CCAGGGAGGGGAGAAATACTTTTTGGCTGACTCTCCTGACTCTGTGTTT
CCTACAATTCTACAACAAAGATCCAGGAACATGAGAAAAGGAAAATGAG
AAAATGAGT;
for POU3F1:
(SEQ ID NO: 2)
CATTGGCTGCCCGCGGAGCTGCCTCCCGCCTCCCCTGCCCGGCCCCCGC
CCCGCGCGCCCGCCCGCCACCCTGCCCGGCCGCCCAGAGCTCTCCATT;
and
for YPEL3:
(SEQ ID NO: 3)
AGGGAGTAGGTGGGCTGTCAGGACCTGGGCCACACACATGCGAGGCACT
CCCAGAGCCGTGGGGACTCGCTCTGTCACACTGGGCTGCTCTCTCCTTT
CCCCAGAGCCAGCAGCCTCTCCGGGGACCAGAGGCGTCTCGGTTTTGA
C.
for MAFB:
(SEQ ID NO: 4)
CGCTCGCAGCCGCTCGCAGCTCGGCGGTGCAGCTGTGCTGGATCCGGCG
GCGCCGCAGCCTTTTATCGCCTCCTGATGTCACTGGGGTGCGGGGGCCC
GGGCGGCCCGGTGCGCGGGCCAATAGCTGCACGGCCTCCGCGGCCCAGC
GG.

In certain samples, increased methylation of cg6522, POU3F1, YPEL3, and MAFB indicate the presence of HGD, EAC, or JCA. Increased methylation can be represented or indicated by a “methylation value” (e.g., representing a methylation frequency, fraction, ratio, percent, etc.). In certain examples, a methylation value, represents the methylation status and can thus be used as a quantitative indicator of methylation status across multiple copies of a locus. This is of particular use when it is desirable to compare the methylation status of a sequence in a sample to a threshold or reference value. As used herein, “methylation frequency” or “methylation percent (%)” refer to the number of instances in which a molecule or locus is methylated relative to the number of instances the molecule or locus is unmethylated. Certain embodiments disclosed herein utilize a relative methylation percentage (RM %) which is a ratio percent of the amount of target methylated alleles.

While in certain examples, the disclosure provides that increased methylation is indicative of HGD, EAC, or JCA, one of ordinary skill in the art will understand that different reference points can lead to different directional changes indicative of HGD, EAC, or JCA. For example, one may collect a data set of methylation levels of cg6522, POU3F1, YPEL3, and MAFB from patients with HGD, EAC, or JCA. If this data set were used as the reference level, then HGD, EAC, or JCA would be indicated if there was no significant difference from the reference level. An increase in methylation is indicative of HGD, EAC, or JCA when the reference level is in relation to normal or BE samples.

The current disclosure provides that ultra-sensitive methylation-specific droplet digital PCR assays were designed for each individual marker, and their performance was assessed in an initial training set of esophageal biopsy samples. Using Receiver Operating Characteristics (ROC) analysis, the sensitivity and specificity of each biomarker was determined when used singly and in combination as an assay panel. Using a linear regression model, the best performing 2-marker panel was identified, displaying a statistically significant high specificity for normal squamous tissue and NDBE at 92.6% and 83.8%, with a sensitivity of 67% and 82.5% for detection of HGD, EAC, or JCA, respectively in a validation study, which was conducted using an independent set of samples of endoscopy-directed esophageal brushings.

Droplet digital PCR (ddPCR), described in more detail below, is a technology that enables the precise and sensitive detection and absolute quantification of nucleic acid targets in various clinical specimens. Its performance is superior over conventional MethyLight PCR for DNA methylation studies (Weisenberger et al., Nucleic Acids Res 2008; 36:4689-98; Yu, Heinzerling, & Grady, Methods Mol Biol 2018; 1768:363-383; Yu et al., Epigenetics 2015; 10:803-9; Wiencke et al., Epigenetics 2014; 9:1360-5; Van Wesenbeeck et al., Epigenetics 2018; 13:207-213). This disclosure describes the development of methylation-specific ddPCR assays for the top candidate CpG sites from the HM450 array studies. Methylation-specific droplet digital polymerase chain reaction (MS-ddPCR) assays accurately quantify methylated cg6522, YPEL3, POU3F1, and MAFB in as little as 4 ng of DNA from esophageal brushing samples. Since cytologic sampling yields limited amount of DNA with mixed cell types, disclosed results support the use of MS-ddPCR based assays for the development of DNA methylation-based molecular cytology assays for HGD, EAC, and JCA detection.

The strength of this best-performing biomarker panel lies in its capability to differentiate HGD, EAC, and JCA from normal and non-dysplastic BE. If combined with promising BE markers identified and validated, these markers can be used in an esophageal cytology based approach for BE screening and surveillance. Given the emerging technical advances in swallowable cytology collection devices (Kaz & Grady, Transl Gastroenterol Hepatol 2019; 4:25), the DNA-methylation based molecular assays will be further validated in cytology balloon collected samples as this sample collection method appears to have a high likelihood of near-term adoption into clinical practice.

In summary, the disclosure provides the development of DNA-methylation based molecular assays for the detection of HGD/EAC/JCA using cytology samples, which, in certain examples, can be used for the surveillance of BE patients.

Aspects of the current disclosure are now described with additional detail and options, as follows: (i) Epigenetic Biomarker Groupings; (ii) Comparisons & Reference Levels; (iii) Methylation Detection Assays; (iv) Droplet Digital™ PCR (ddPCR™); (v) Kits; (vi) Methods of Use; (vii) Exemplary Embodiments; (viii) Experimental Examples; and (ix) Closing Paragraphs. These headings are provided for organizational purposes only and do not limit the scope or interpretation of the disclosure.
(i) Epigenetic Biomarker Groupings. In particular embodiments, reliable identification of specific cytosine loci distributed throughout the genome has been detailed in, for example, the document “CpG Loci Identification. A guide to Ilumina's method for unambiguous CpG loci identification and tracking for the GOLDENGATE® and INFINIUM® assays for Methylation”. Briefly, Illumina has developed a CpG locus identifier that designates cytosine loci based on the actual or contextual sequence of nucleotides in which the cytosine is located. It uses a similar strategy as used by NCBI's re SNP IPS (rs #) and is based on the sequence flanking the cytosine of interest. Thus a unique CpG locus cluster ID number is assigned to each of the cytosine undergoing evaluation. The system is consistent and not affected by changes in public databases and genome assemblies. Flanking sequences of 60 bases 5′ and 3′ to the CG locus (i.e. a total of 122 base sequences) is used to identify the locus. Thus a unique “CpG cluster number” or cg # is assigned to the sequence of 122 bp which contains the CpG of interest. Thus, only if the 122 bp in the CpG cluster is identical is there a risk of a locus being assigned the same number and being located in more than one position in the genome. Three separate criteria are utilized to track an individual CpG locus based on this unique ID system, chromosome number, genomic coordinate, and genome build. The lesser of the two coordinates “C” or “G” in CpG is used in the unique CG loci identification. The CG locus is also designated in relation to the first ‘unambiguous” pair of nucleotides containing either an ‘A’ or ‘T’. If one of these nucleotides is 5′ to the CG then the arrangement is designated TOP and if such a nucleotide is 3′ it is designated BOT.

The current disclosure describes use of the methylation status of cg6522 (cg41566522), POU3F1 (cg38512601), YPEL3 (cg16348385), and/or MAFB (cg18251612) to characterize a subject as having normal or Barrett's esophagus as compared to having high grade dysplasia (HGD), esophageal adenocarcinoma (EAC), or junctional adenocarcinoma (JCA).

Particular embodiments utilize methylation status of all four marker genes: cg6522, POU3F1, YPEL3, and MAFB. Particular embodiments utilize methylation status of three marker genes: cg6522, POU3F1, and YPEL3; cg6522, POU3F1, and MAFB; cg6522, YPEL3, and MAFB; or POU3F1, YPEL3, and MAFB. Other embodiments utilize the two-marker panel: cg6522 and POU3F1. Particular embodiments can also utilize cg6522 and YPEL3; cg6522, and MAFB; POU3F1 and YPEL3; POU3F1 and MAFB; or YPEL3, and MAFB.

Particular embodiments may also actively exclude use of a marker gene. For example, certain embodiments exclude use of cg6522. Certain embodiments exclude use of POU3F1. Certain embodiments exclude use of YPEL3. Certain embodiments exclude use of MAFB. Certain embodiments exclude use of any marker other than cg6522, POU3F1, YPEL3, and MAFB. Other embodiments exclude use of any marker other than cg6522 and POU3F1.

In particular embodiments when more than one marker is assayed, values of the detected markers can be calculated into a score. Each value can be weighted evenly within an algorithm generating a score, or the values for particular markers can be weighted more heavily in reaching the score. For example, markers with higher AUC or pAUC and/or methylation difference scores could be weighted more heavily than markers with lower AUC or pAUC and/or methylation difference scores. For example, in particular embodiments, cg6522 and POU3F1 may be weighted more heavily than YPEL3 or MAFB.

Markers may also be grouped into classes, and each class given a weighted score. For example, marker values for distinguishing normal or Barrett's esophagus from HGD, EAC, or JCA may be grouped into classes and weighted as follows (from highest weight to lowest weight): Class 1: cg6522 and POU3F1; Class 2: YPEL3; and Class 3: other validated markers including MAFB.

Any marker or class of markers can be included in a particular value calculation. For example, in particular embodiments, Class 1 is included. In particular embodiments, Class 2 is included. In particular embodiments, Class 3 is included. In particular embodiments, groups of classes can be included, for example, Classes 1 and 2; 1 and 3; and/or 2 and 3. Particular classes can also be excluded. For example, in particular embodiments, Class 1 is excluded. In particular embodiments, Class 2 is excluded. In particular embodiments, Class 3 is excluded.
(ii) Comparisons & Reference Levels. As indicated previously, up (hyper)- or down (hypo)-methylation of the markers (e.g., methylation status) can be assessed and a methylation value can be generated and compared to a relevant reference level. The value can be one or more numerical values resulting from the assaying of a sample, and can be derived, e.g., by measuring methylation status of the marker(s) in the sample by an assay, or from a dataset obtained from a provider such as a laboratory, or from a dataset stored on a server.

In the broadest sense, the value may be qualitative or quantitative. As such, where detection is qualitative, the methods and kits provide a reading or evaluation, e.g., assessment, of whether or not the marker is methylated in the sample being assayed. In further embodiments, the methods and kits provide a quantitative detection of methylation, i.e., an evaluation or assessment of the actual amount or relative abundance of methylation of the marker in the sample being assayed. In such embodiments, the quantitative detection may be absolute or relative. As such, the term “quantifying” when used in the context of quantifying methylation of a marker in a sample can refer to absolute or to relative quantification. Absolute quantification can be accomplished by inclusion of samples with known methylation parameters as one or more control markers and referencing, e.g., normalizing, the detected methylation level of the experimental marker with the known control markers (e.g., through generation of a standard curve). Alternatively, relative quantification can be accomplished by comparison of generated methylation values between two or more different markers to provide a relative quantification of each of the two or more markers, e.g., relative to each other. The actual measurement of values for the markers can be determined using any method known in the art.

As stated previously, detected marker levels (e.g., methylation values) can be compared to one or more reference levels. Reference levels can be obtained from one or more relevant datasets. A “dataset” as used herein is a set of numerical values resulting from evaluation of a sample (or population of samples) under a desired condition. The values of the dataset can be obtained, for example, by experimentally obtaining measures from sample(s) and constructing a dataset from these measurements. As is understood by one of ordinary skill in the art, the reference level can be based on e.g., any mathematical or statistical formula useful and known in the art for arriving at a meaningful aggregate reference level from a collection of individual datapoints; e.g., mean, median, median of the mean, etc. Alternatively, a reference level or dataset to create a reference level can be obtained from a service provider such as a laboratory, or from a database or a server on which the dataset has been stored.

A reference level from a dataset can be derived from previous measures derived from a population. A “population” is any grouping of subjects or samples of like specified characteristics. The grouping could be according to, for example, clinical parameters, clinical assessments, therapeutic regimens, disease status, severity of HGD, EAC, or JCA, etc. In particular embodiments, a population is a group of subjects with normal or Barrett's esophagus. In particular embodiments, a population is a group of subjects with HGD, EAC, or JCA.

In particular embodiments, conclusions are drawn based on whether a methylation value is statistically significantly different or not statistically significantly different from a reference level. A measure is not statistically significantly different if the difference is within a level that would be expected to occur based on chance alone. In contrast, a statistically significant difference is one that is greater than what would be expected to occur by chance alone. Statistical significance or lack thereof can be determined by any of various methods well-known in the art. An example of a commonly used measure of statistical significance is the p-value. The p-value represents the probability of obtaining a given result equivalent to a particular datapoint, where the datapoint is the result of random chance alone. A result is often considered significant (not random chance) at a p-value less than 0.05.

In particular embodiments, methylation values obtained based on the markers and/or other dataset components can be subjected to an analytic process with chosen parameters. The parameters of the analytic process may be those disclosed herein or those derived using the guidelines described herein. The analytic process used to generate a result may be any type of process capable of distinguishing normal or Barrett's esophageal samples from HGD, EAC, or JCA samples based on methylation status detection, for example, a linear algorithm, a quadratic algorithm, a decision tree algorithm, or a voting algorithm. The analytic process may set a threshold for determining the probability that a sample belongs to a given class. The probability preferably is at least 60%, at least 70%, at least 80%, at least 90%, at least 95% or higher. Detection relies on performing a methylation assay on the biological sample.

The receiver operating characteristics (ROC) curve is a graph plotting sensitivity (true positive rate), which is defined in this setting as the percentage of HGD, EAC, or JCA cases with a positive test on the Y axis and false positive rate (1-specificity), i.e. the number of normal or Barrett's esophagus cases with a positive test on the X-axis. False positive rate refers to the percentage of normal or Barrett's esophagus subjects falsely found to have a positive test.

The area under the ROC curves (AUC) indicates the accuracy of the test in identifying normal from abnormal cases (Hanley & McNeil, Radiology 1982; 143:29-36). The AUC is the area under the ROC plot from the curve to the diagonal line from the point of intersection of the X- and Y-axes and with an angle of incline of 45° (a test with no discrimination between two groups, e.g., normal or Barrett's esophagus vs. HGD, EAC, or JCA), has a 45° diagonal line from the lower left to the upper right corner). The higher the area under the receiver operating characteristics (ROC) curve, the greater the accuracy of the test in predicting the condition of interest. An area ROC=1.0 indicates a perfect test, which is positive in all cases with the disorder (e.g, HGD, EAC, or JCA) and negative in all normal individuals without the disorder (e.g., normal or Barrett's esophagus). Thus, the closer the plot is to the upper left corner, the higher the overall accuracy of the test.

Certain embodiments disclosed herein utilize logistic regression models. In some embodiments, the method includes utilizing a logistic regression model (e.g., as described in the “Examples” section of the present application) to categorize a sample as a normal or BE sample versus an HGD, EAC or JCA samples. In certain examples, a logistic regression model can be trained on biopsy sample sets (training set) as

$$log it ( p ) = log ( p / ( 1 - p ) ) = {β}_{0} + {β}_{1} {x}_{1} + … + {β}_{n} {x}_{n} ,$$

where p and β stand for the probability score of the specified sample being positive and the intercept or coefficient of each marker.
(iii) Methylation Detection Assays. Methylation of the markers can be assessed using various methylation detection assays. A “methylation detection assay” refers to an assay, which can be commercially available, for distinguishing methylated versus unmethylated cytosine loci in DNA. Techniques for measuring cytosine methylation include bisulfite-based methylation assays. The addition of bisulfite to DNA results in the methylation of unmethylated cytosine and its ultimate conversion to the nucleotide uracil. Uracil has similar binding properties to thiamine in the DNA sequence. Previously methylated cytosine does not undergo similar chemical conversion on exposure to bisulfite. Bisulfite assays can thus be used to discriminate previously methylated versus unmethylated cytosine.

Within the current disclosure, a bisulfite converted cg6522 assay amplicon includes:

(SEQ ID NO: 5)
AATGTAGTCGGTTTTGCGTATGTGGTGTTTGAGTAGGGTTCGAGATTTT
TTAGGGAGGGGAGAAATATTTTTTGGTTGATTTTTTTGATTTTGTGTTT
TTTATAATTTTATAATAAAGATTTAGGAATATGAGAAAAGGAAAATGAG
AAAATGAGT;

a bisulfite converted POU3F1 assay amplicon includes:

(SEQ ID NO: 6)
TATTGGTTGTTCGCGGAGTTGTTTTTCGTTTTTTTTGTTCGGTTTTCGT
TTCGCGCGTTCGTTCGTTATTTTGTTCGGTCGTTTAGAGTTTTTTATT;
and

a bisulfite converted YPEL3 assay amplicon includes:

(SEQ ID NO: 7)
AGGGAGTAGGTGGGTTGTTAGGATTTGGGTTATATATATGCGAGGTATT
TTTAGAGTCGTGGGGATTCGTTTTGTTATATTGGGTTGTTTTTTTTTTT
TTTTAGAGTTAGTAGTTTTTTCGGGGATTAGAGGCGTTTCGGTTTTGA
T.

a bisulfite converted MAFB1612 assay amplicon includes:

(SEQ ID NO: 8)
CCGCTAAACCGCGAAAACCGTACAACTATTAACCCGCGCACCGAACCGC
CCGAACCCCCGCACCCCAATAACATCAAAAAACGATAAAAAACTACGAC
GCCGCCGAATCCAACACAACTACACCGCCGAACTACGAACGACTACGAA
CG.

An exemplary quantitative methylation detection assay combines bisulfite treatment and restriction analysis COBRA, which uses methylation sensitive restriction endonucleases, gel electrophoresis, and detection based on labeled hybridization probes. (Ziong and Laird, Nucleic Acid Res. 1997 25; 2532-4). Another exemplary detection assay is the methylation specific polymerase chain reaction PCR (MSPCR) for amplification of DNA segments of interest. This assay can be performed after sodium bisulfite conversion of cytosine and uses methylation sensitive probes. Other detection assays include the Quantitative Methylation (QM) assay, which combines PCR amplification with fluorescent probes designed to bind to putative methylation sites; MethyLight™ (Qiagen, Redwood City, CA) a quantitative methylation detection assay that uses fluorescence based PCR (Eads, et al., Cancer Res. 1999; 59:2302-2306); and Ms-SNuPE, a quantitative technique for determining differences in methylation levels in CpG sites. As with other techniques, Ms-SNuPE also requires bisulfite treatment to be performed first, leading to the conversion of unmethylated cytosine to uracil while methyl cytosine is unaffected. PCR primers specific for bisulfite converted DNA are then used to amplify the target sequence of interest (see Table 1). The amplified PCR product is isolated and used to quantitate the methylation status of the CpG site of interest. (Gonzalgo and Jones Nuclei Acids Res 1997; 25:252-31).

In particular embodiments, the INFINIUM® (Ilumina, Inc., San Diego California, USA) Human Methylation 450 Beadchip assay can be used. The Illumina assay can be used for genome wide quantitative methylation profiling. In particular embodiments, genomic DNA can be extracted from cells. Genomic DNA can be isolated and proteins or other contaminants can be removed from the DNA using proteinase K. The DNA can then be removed from the solution using available methods such as organic extraction, salting out, or binding the DNA to a solid phase support. As described above, and in the Infinium® Assay Methylation Protocol Guide, the DNA can be treated with sodium bisulfite. The bisulfite converted DNA can then be denatured and amplified. A next step can use enzymatic means to fragment the DNA. The fragmented DNA can then be precipitated using isopropanol and separated by centrifugation. The separated DNA can next be suspended in a hybridization buffer. The fragmented DNA can then be hybridized to beads that have been covalently limited to 50mer nucleotide segments at a locus specific to the cytosine nucleotide of interest in the genome. There are a total of over 500,000 bead types specifically designed to anneal to the locus where the particular cytosine is located, and the beads are bound to silicon-based arrays. There are two bead types designed for each locus, one bead type represents a probe that is designed to match to the methylated locus at which the cytosine nucleotide will remain unchanged. The other bead type corresponds to an initially unmethylated cytosine, which after sodium bisulfite treatment, is converted to uracil and ultimately a thiamine nucleotide. Unhybridized DNA (DNA not annealed to the beads) is washed away leaving only DNA segments bound to the appropriate bead and containing the cytosine of interest. If the cytosine of interest was unmethylated prior to the sodium bisulfite treatment, then it will match with the unmethylated or “U” bead probe. If the cytosine was methylated, single base mismatch will occur with the “U” bead probe oligomer. No further nucleotide extension on the bead oligomer occurs. This will lead to low fluorescent signal from the “U” bead. The reverse will happen on the “M” or methylated bead probe.

Lasers can then be used to stimulate fluorophores bound to the beads. The level of methylation at each cytosine locus is detected by the intensity of the fluorescence from the methylated compared to the unmethylated bead. Cytosine methylation level is expressed as “B” which is the ratio of the methylated-bead probe signal to total signal intensity at that cytosine locus.

In particular embodiments, pyrosequencing can be used to detect marker methylation. Pyrosequencing is a method of DNA sequencing that relies on detection of the release of pyrophosphates as DNA is synthesized (and is therefore a “sequencing by synthesis” technique). To assess methylation by pyrosequencing, a DNA sample can be incubated with sodium bisulfite, converting unmethylated cytosine to uracil. The presence of uracil will result in thymine incorporation during PCR amplification. Therefore, sequencing results that include thymine at a nucleotide position that is known to encode cytosine can be interpreted as unmethylated sites. In contrast cytosines present in the sequencing results indicate that the site was methylated in the original DNA sample, because methylation protects cytosine from conversion to uracil upon treatment. Bisulfite treatment can also be performed on control samples with known methylation patterns, to reduce or eliminate false positive results. Commercially available pyrosequencing machines include Pyro Mark Q96 (Qiagen, Hilden, Germany). For more details on methods to use pyrosequencing for measurement of methylation, see Delaney et al. Methods Mol Biol. 2015 1343:249-264. Pyrosequencing is especially useful for detecting methylation in the CpG sites within genes.

While not preferred, measurement of mRNA levels transcribed by genes with altered cytosine methylation can also be assessed to indirectly indicate methylation status. Any technique for determining expression levels of mRNA can be used including Northern blot analysis, fluorescent in situ hybridization (FISH), RNase protection assays (RPA), microarrays, PCR-based, or other technologies for measuring RNA levels can be used.

Up (hyper)- or down (hypo)-methylation of genes also can be detected indirectly using, for example, cDNA arrays, cDNA fragment fingerprinting, cDNA sequencing, clone hybridization, differential display, differential screening, FRET detection, liquid microarrays, PCR, RT-PCR, quantitative RT-PCR analysis with TaqMan assays, molecular beacons, microelectric arrays, oligonucleotide arrays, polynucleotide arrays, serial analysis of gene expression (SAGE), and/or subtractive hybridization.

Further hybridization technologies that may be used are described in, for example, U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; and 5,800,992 as well as WO 95/21265; WO 96/31622; WO 97/10365; WO 97/27317; EP 373 203; and EP 785 280.

Additionally, protein products of genes that are differentially methylated can be measured to indirectly assess cytosine methylation levels. Proteins translated from mRNA reflect the same phenomenon of altered gene expression related to changes in cytosine methylation. Therefore, protein expression could also be used to biologically classify a sample as normal or Barrett's esophageal versus HGD, EAC, or JCA.

“Protein detection” includes detection of full-length proteins, mature proteins, pre-proteins, polypeptides, isoforms, mutations, post-translationally modified proteins and variants thereof, and can be detected in any suitable manner.

In particular embodiments, a protein marker is detected by contacting a sample with reagents (e.g., antibodies), generating complexes of reagent and marker(s), and detecting the complexes. Particular embodiments for detecting and measuring protein levels can use methods including agglutination, chemiluminescence, electro-chemiluminescence (ECL), enzyme-linked immunoassays (ELISA), immunoassay, immunoblotting, immunodiffusion, immunoelectrophoresis, immunofluorescence, immunohistochemistry, immunoprecipitation, mass-spectrometry, and western blot. See also, e.g., E. Maggio, Enzyme-Immunoassay (1980), CRC Press, Inc., Boca Raton, Fla; and U.S. Pat. Nos. 4,727,022; 4,659,678; 4,376,110; 4,275,149; 4,233,402; and 4,230,797.

Various methylation detection assays use nucleic acids and/or proteins linked to chips, such as microarray chips. See, for example, U.S. Pat. Nos. 5,143,854; 6,087,112; 5,215,882; 5,707,807; 5,807,522; 5,958,342; 5,994,076; 6,004,755; 6,048,695; 6,060,240; 6,090,556; and 6,040,138. Binding to nucleic acids or proteins on microarrays can be detected by scanning the microarray with a variety of lasers or charge coupled device (CCD)-based scanners, and extracting features with software packages, for example, Imagene (Biodiscovery, Hawthorne, CA), Feature Extraction Software (Agilent), Scanalyze (Eisen, M. 1999. SCANALYZE User Manual; Stanford Univ., Stanford, Calif. Ver 2.32.), or GenePix (Axon Instruments).

Embodiments disclosed herein can be used with high throughput screening (HTS). Typically, HTS refers to a format that performs at least 100 assays, at least 500 assays, at least 1000 assays, at least 5000 assays, at least 10,000 assays, or more per day. When enumerating assays, either the number of samples or the number of markers assayed can be considered.

Generally, HTS methods involve a logical or physical array of either samples, or the nucleic acid or protein markers, or both. Appropriate array formats include both liquid and solid phase arrays. For example, assays employing liquid phase arrays, e.g., for hybridization of nucleic acids, binding of antibodies or other receptors to ligand, etc., can be performed in multiwell or microtiter plates. Microtiter plates with 96, 384, or 1536 wells are widely available, and even higher numbers of wells, e.g., 3456 and 9600 can be used. In general, the choice of microtiter plates is determined by the methods and equipment, e.g., robotic handling and loading systems, used for sample preparation and analysis.

HTS assays and screening systems are commercially available from, for example, Zymark Corp. (Hopkinton, MA); Air Technical Industries (Mentor, OH); Beckman Instruments, Inc. (Fullerton, CA); Precision Systems, Inc. (Natick, MA), etc. These systems typically automate entire procedures including all sample and reagent pipetting, liquid dispensing, timed incubations, and final readings of the microplate in detector(s) appropriate for the assay. These configurable systems provide HTS as well as a high degree of flexibility and customization. The manufacturers of such systems provide detailed protocols for the various methods of HTS.
(iv) Droplet Digital™ PCR (ddPCR™). Particular embodiments utilize ddPCR™ (Bio-Rad Laboratories, Hercules, CA). ddPCR technology uses a combination of microfluidics and surfactant chemistry to divide PCR samples into water-in-oil droplets. Hindson et al., Anal. Chem. 83(22): 8604-8610 (2011). The droplets support PCR amplification of the target template molecules they contain and use reagents and workflows similar to those used for most standard Taqman probe-based assays. For example, within the context of the current disclosure, a representative droplet would include fragmented DNA for analysis and primer/probe sequences from the table below (or sequences having at least 90% sequence identity thereto):

TABLE 1
The primer and probe sequences for the methylation specific droplet
digital PCR assays

| Assay | Primer/Probe | Sequence 5′→3′ |
|---|---|---|
| cg6522 | Forward | AATGTAGTCGGTTTTGCGTATGTG (SEQ ID NO: 9) |
| | Reverse | ACTCATTTTCTCATTTTCCTTTTCTCA (SEQ ID NO: 10) |
| | Probe | FAM-AGTAGGGTTCGAGATTT-MGBNFQ* (SEQ ID NO: 11) |
| YPEL3 | Forward | AGGGAGTAGGTGGGTTGTTAGGAT (SEQ ID NO: 12) |
| | Reverse | ATCAAAACCGAAACGCCTCTAAT (SEQ ID NO: 13) |
| | Probe | FAM-TAGAGTCGTGGGGATTC-MGBNFQ* (SEQ ID NO: 14) |
| POU3F1 | Forward | TATTGGTTGTTCGCGGAGTTG (SEQ ID NO: 15) |
| | Reverse | AATAAAAAACTCTAAACGACCGAACAA (SEQ ID NO: 16) |
| | Probe | FAM-TTTCGCGCGTTCGTT-MGBNFQ* (SEQ ID NO: 17) |
| MAFB | Forward | CCGCTAAACCGCGAAAAC (SEQ ID NO: 18) |
| | Reverse | CGTTCGTAGTCGTTCGTAGTTCG (SEQ ID NO: 19) |
| | Probe | FAM-ACTACGACGCCGCCG-MGBNFQ* (SEQ ID NO: 20) |

*MGBNFQ refers to Minor Groove Binding Nonfluorescent Quencher in the 3′ terminus of probe
Following PCR, each droplet is analyzed or read in a flow cytometer to determine the fraction of PCR-positive droplets in the original sample. These data are then analyzed using Poisson statistics to determine the target concentration in the original sample. See Bio-Rad Droplet Digital ™ (ddPCR ™) PCR Technology.

While ddPCR™ is a preferred approach utilized within the current disclosure, as indicated above, other sample partition PCR methods based on the same underlying principles may also be used. These approaches are now described more generally.

Sample Partitioning. Numerous methods can be used to divide samples into discrete partitions (e.g., droplets). Exemplary partitioning methods and systems include use of one or more of emulsification, droplet actuation, microfluidics platforms, continuous-flow microfluidics, reagent immobilization, and combinations thereof. In some embodiments, partitioning is performed to divide a sample into a sufficient number of partitions such that each partition contains one or zero nucleic acid molecules. In some embodiments, the number and size of partitions is based on the concentration and volume of the bulk sample.

Methods and devices for partitioning a bulk volume into partitions by emulsification are described in Nakano et al. J. Biotechnol. 102, 117-124 (2003) and Margulies et al. Nature 437, 376-380 (2005). Systems and methods to generate “water-in-oil” droplets are described in U.S. Publication No. 2010/0173394. Microfluidics systems and methods to divide a bulk volume into partitions are described in U.S. Publication Nos. 2010/0236929; 2010/0311599; and 2010/0163412, and U.S. Pat. No. 7,851,184. Microfluidic systems and methods that generate monodisperse droplets are described in Kiss et al. Anal. Chem. 80(23), 8975-8981 (2008). Further microfluidics systems and methods for manipulating and/or partitioning samples using channels, valves, pumps, etc. are described in U.S. Pat. No. 7,842,248. Continuous-flow microfluidics systems and methods are described in Kopp et al., Science, 280, 1046-1048 (1998).

Partitioning methods can be augmented with droplet manipulation techniques, including electrical (e.g., electrostatic actuation, dielectrophoresis), magnetic, thermal (e.g., thermal Marangoni effects, thermocapillary), mechanical (e.g., surface acoustic waves, micropumping, peristaltic), optical (e.g., opto-electrowetting, optical tweezers), and chemical means (e.g., chemical gradients). In some embodiments, a droplet microactuator is supplemented with a microfluidics platform (e.g. continuous flow components).

Some embodiments use a droplet microactuator. A droplet microactuator can be capable of effecting droplet manipulation and/or operations, such as dispensing, splitting, transporting, merging, mixing, agitating, and the like. Droplet operation structures and manipulation techniques are described in U.S. Publication Nos. 2006/0194331 and 2006/0254933 and U.S. Pat. Nos. 6,911,132; 6,773,566; and 6,565,727.

Amplification. The partitioned nucleic acids of a sample can be amplified by any suitable PCR methodology that can be practiced with methylation-based PCR assays. Exemplary PCR types include allele-specific PCR, assembly PCR, asymmetric PCR, endpoint PCR, hot-start PCR, in situ PCR, intersequence-specific PCR, inverse PCR, linear after exponential PCR, ligation-mediated PCR, methylation-specific PCR, miniprimer PCR, multiplex ligation-dependent probe amplification, multiplex PCR, nested PCR, overlap-extension PCR, polymerase cycling assembly, qualitative PCR, quantitative PCR, real-time PCR, single-cell PCR, solid-phase PCR, thermal asymmetric interlaced PCR, touchdown PCR, universal fast walking PCR, etc. Ligase chain reaction (LCR) may also be used.

PCR may be performed with a thermostable polymerase, such as Taq DNA polymerase (e.g., wild-type enzyme, a Stoffel fragment, FastStart polymerase, etc.), Pfu DNA polymerase, S-Tbr polymerase, Tth polymerase, Vent polymerase, or a combination thereof, among others.

PCR are driven by thermal cycling. Alternative amplification reactions, which may be performed isothermally, can also be used. Exemplary isothermal techniques include branched-probe DNA assays, cascade-RCA, helicase-dependent amplification, loop-mediated isothermal amplification (LAMP), nucleic acid based amplification (NASBA), nicking enzyme amplification reaction (NEAR), PAN-AC, Q-beta replicase amplification, rolling circle replication (RCA), self-sustaining sequence replication, strand-displacement amplification, etc.

Amplification may be performed with any suitable reagents (e.g. template nucleic acid (e.g. DNA or RNA)), primers, probes, buffers, replication catalyzing enzymes (e.g. DNA polymerase, RNA polymerase), nucleotides, salts (e.g. MgCl₂), etc. In some embodiments, an amplification mixture includes any combination of at least one primer or primer pair, at least one probe, at least one replication enzyme (e.g., at least one polymerase), and deoxynucleotide (and/or nucleotide) triphosphates (dNTPs and/or NTPs), etc.

Amplification reagents can be added to a sample prior to partitioning, concurrently with partitioning and/or after partitioning has occurred. In some embodiments, all partitions are subjected to amplification conditions (e.g. reagents and thermal cycling), but amplification only occurs in partitions containing target nucleic acids (e.g. nucleic acids containing sequences complementary to primers added to the sample). The template nucleic acid can be the limiting reagent in a partitioned amplification reaction. In some embodiments, a partition contains one or zero target (e.g. template) nucleic acid molecules.

As indicated previously, in some embodiments, nucleic acid targets, primers, and/or probes are immobilized to a surface, for example, a substrate, plate, array, bead, particle, etc. Immobilization of one or more reagents provides (or assists in) one or more of: partitioning of reagents (e.g. target nucleic acids, primers, probes, etc.), controlling the number of reagents per partition, and/or controlling the ratio of one reagent to another in each partition. In some embodiments, assay reagents and/or target nucleic acids are immobilized to a surface while retaining the capability to interact and/or react with other reagents (e.g. reagent dispensed from a microfluidic platform, a droplet microactuator, etc.). In some embodiments, reagents are immobilized on a substrate and droplets or partitioned reagents are brought into contact with the immobilized reagents. Techniques for immobilization of nucleic acids and other reagents to surfaces are well understood by those of ordinary in the art. See, for example, U.S. Pat. No. 5,472,881 and Taira et al. Biotechnol. Bioeng. 89(7), 835-8 (2005).

Target Sequence Detection. Detection methods can be utilized to identify sample partitions containing amplified target(s). Detection can be based on one or more characteristics of a sample partition such as a physical, chemical, luminescent, or electrical aspects, which correlate with amplification.

In particular embodiments, fluorescence detection methods are used to detect amplified target(s), and/or identification of sample partitions containing amplified target(s). Exemplary fluorescent detection reagents include TaqMan probes, SYBR Green fluorescent probes, molecular beacon probes, scorpion probes, and/or LightUp Probes® (LightUp Technologies AB, Huddinge, Sweden). Additional detection reagents and methods are described in, for example, U.S. Pat. Nos. 5,945,283; 5,210,015; 5,538,848; and 5,863,736; PCT Publication WO 97/22719; and publications: Gibson et al., Genome Research, 6, 995-1001 (1996); Heid et al., Genome Research, 6, 986-994 (1996); Holland et al., Proc. Natl. Acad. Sci. USA 88, 7276-7280, (1991); Livak et al., Genome Research, 4, 357-362 (1995); Piatek et al., Nat. Biotechnol. 16, 359-63 (1998); Neri et al., Advances in Nucleic Acid and Protein Analysis, 3826, 117-125 (2000); Compton, Nature 350, 91-92 (1991); Thelwell et al., Nucleic Acids Research, 28, 3752-3761 (2000); Tyagi and Kramer, Nat. Biotechnol. 14, 303-308 (1996); Tyagi et al., Nat. Biotechnol. 16, 49-53 (1998); and Sohn et al., Proc. Natl. Acad. Sci. U.S.A. 97, 10687-10690 (2000).

In some embodiments, detection reagents are included with amplification reagents added to the bulk or partitioned sample. In some embodiments, amplification reagents also serve as detection reagents. In some embodiments, detection reagents are added to partitions following amplification. In some embodiments, measurements of the absolute copy number and the relative proportion of target nucleic acids in a sample (e.g. relative to other targets nucleic acids, relative to non-target nucleic acids, relative to total nucleic acids, etc.) can be measured based on the detection of sample partitions containing amplified targets.

In some embodiments, following amplification, sample partitions containing amplified target(s) are sorted from sample partitions not containing amplified targets or from sample partitions containing other amplified target(s). In some embodiments, sample partitions are sorted following amplification based on physical, chemical, and/or optical characteristics of the sample partition, the nucleic acids therein (e.g. concentration), and/or status of detection reagents. In some embodiments, individual sample partitions are isolated for subsequent manipulation, processing, and/or analysis of the amplified target(s) therein. In some embodiments, sample partitions containing similar characteristics (e.g. same fluorescent labels, similar nucleic acid concentrations, etc.) are grouped (e.g. into packets) for subsequent manipulation, processing, and/or analysis.

(v) Kits. Kits disclosed herein include materials to assay a sample for the methylation status of one or more markers disclosed herein. In particular embodiments, the kits include materials to conduct PCR. Materials to conduct PCR include components of amplification mixtures, such as at least one primer or primer pair, at least one probe, at least one replication enzyme (e.g., at least one polymerase), and deoxynucleotide (and/or nucleotide) triphosphates (dNTPs and/or NTPs), etc. Particular embodiments provide the primer and probes sequences provided in Table 1 or sequences having at least 90% sequence identity thereto. Kits can also include bisulfite.

Additional embodiments include detection reagents. Exemplary detection reagents can include radioactive isotopes or radiolabels (e.g., 32P and 13C), enzymes (e.g., luciferase, HRP and AP), dyes (e.g., rhodamine and cyanine), fluorescent tags or dyes (e.g., GFP, YFP, FITC), magnetic beads, or biotin. In particular embodiments, the detectable label is fluorescein, GFP, rhodamine, cyanine dyes, Alexa dyes, luciferase, or a radiolabels. TaqMan probes, SYBR Green fluorescent probes, molecular beacon probes, scorpion probes, and/or LightUp Probes® (LightUp Technologies) may also be used.

Particular embodiments can include reference levels and/or control conditions (positive and/or negative).

Instructions for carrying out and interpreting methylation assays, including, optionally, instructions for generating a score, can also be included in a kit. Instructions can be provided in written, taped, videoed, VCR, CD-ROM, flashdrive, USB formats or can be provided on a website or other remote location.

In particular embodiments, kits exclude equipment (e.g., plate readers). In particular embodiments, kits exclude materials commonly found in laboratory settings (pipettes; test tubes; distilled H2O).
(vi) Methods of Use. Methods of using methylation-based assays disclosed herein are also provided. Particular embodiments include assaying a sample derived from a subject for the methylation status of disclosed markers or marker combinations.

The assayed sample can be any appropriate biological sample. Particular embodiments utilize cytology samples (e.g., esophageal brushing samples). Certain examples include obtaining samples through endoscopic brushing or nonendoscopic whole esophageal brushing or swabbing using a tethered device (e.g. such as a capsule sponge, balloon, or other device). Particular examples can include obtaining esophageal brushings using a high capacity cytology brush (Hobbs Medical, Stafford Springs, CT) with circumferential sampling from the cardia through the full esophageal length (BE+squamous mucosa) to simulate a swallowed sponge-on-string device. Capsule sponge devices such as those available from PAVMed/Capnostics (Esophacap device) or Medtronic (Cytosponge device) may also be used. In certain examples, after sample acquisition, brushes or sponges can be placed into containers with lysis buffer, and optionally frozen at −80C for later analysis.

In particular embodiments the sample is obtained from a healthy subject, a subject having Barrett's esophagus, a subject having HGD, a subject having EAC, or a subject having JCA.

Particular embodiments disclosed herein include obtaining a sample from a subject having Barrett's esophagus; performing a methylation detection assay on the sample; determining one or more methylation values based on the assaying; and distinguishing Barrett's esophagus from HGD, EAC, or JCA based on the differential methylation value of a marker, as described elsewhere herein.

Particular embodiments also include predicting or diagnosing HGD, EAC, or JCA in a subject by obtaining a sample from a subject suspected of having Barrett's esophagus; assaying the sample for methylation status of one or more markers disclosed herein; generating one or more methylation values based on the assaying; comparing the one or more methylation values to a reference level; and predicting or diagnosing Barrett's esophagus, HGD, EAC, or JCA in the subject according to the methylation value of the marker as determined by the up- or down-methylation of the one or more markers, as described elsewhere herein.

A prediction or diagnosis according to the methods and kits disclosed herein can direct a treatment regimen. For example, a biological classification, prediction or diagnosis of HGD, EAC, or JCA can direct a more aggressive or experimental treatment course. For example, a prediction or diagnosis of HGD, EAC, or JCA can direct a subject to receive RFA or EMR. A classification, prediction or diagnosis of Barrett's esophagus can direct a less aggressive or no further treatment course. Those of ordinary skill in the art classify treatments at a particular time as aggressive, experimental, moderate, minimal or “no” treatment based on a subject's prognosis and relevant standards and treatments at the time. For example, a treatment undergoing a clinical trial is an experimental treatment. Once the treatment is approved by a relevant regulatory agency within a jurisdiction, the treatment is no longer experimental in that jurisdiction.

The Exemplary Embodiments and Examples below are included to demonstrate particular embodiments of the disclosure. Those of ordinary skill in the art should recognize in light of the present disclosure that many changes can be made to the particular embodiments disclosed herein and still obtain a like or similar result without departing from the spirit and scope of the disclosure.

### (vii) Exemplary Embodiments

1. A method to test a subject for the presence of high grade dysplasia (HGD), esophageal adenocarcinoma (EAC), or junctional adenocarcinoma (JCA) in the subject, the method including:
   obtaining a tissue sample derived from the subject;
   detecting a methylation status of cytosines located in an epigenetic loci selected from cg6522, POU3F1, YPEL3, and/or MAFB from the tissue sample to generate a methylation value;
   and
   determining that high grade dysplasia (HGD), esophageal adenocarcinoma (EAC), or junctional adenocarcinoma (JCA) is present or absent in the subject based on the generated methylation value.
2. The method of embodiment 1, wherein the generated methylation value is increased in comparison to a reference level and the HGD, EAC, or JCA is present.
3. The method of embodiments 1 or 2, wherein the generated methylation value is not increased in comparison to a reference level and the HGD, EAC, or JCA is not present.
4. A method to diagnose a subject as having (i) a normal or Barrett's esophagus tissue sample or (ii) a high grade dysplasia (HGD), esophageal adenocarcinoma (EAC), or junctional adenocarcinoma (JCA) tissue sample, the method including:
   obtaining a tissue sample derived from the subject;
   detecting a methylation status of cytosines located in an epigenetic loci selected from cg6522, POU3F1, YPEL3, and/or MAFB from the tissue sample to generate a methylation value;
   and
   diagnosing the subject as having (i) normal or Barrett's esophagus or (ii) HGD, EAC, or JCA based on the generated methylation score.
5. The method of embodiment 4, wherein the subject is diagnosed as having normal or Barrett's esophagus when the generated methylation value is not increased over a reference level.
6. The method of embodiments 4 or 5, wherein the subject is diagnosed as having HGD, EAC, or JCA when the generated methylation value is increased over a reference level.
7. The method of any of embodiments 2, 3, 5, or 6, wherein the reference level is derived from a population of subjects having normal or Barrett's esophagus.
8. The method of any of embodiments 1-7, wherein the epigenetic loci include (i) cg6522, POU3F1, and YPEL3; or (ii) cg6522, POU3F1, YPEL3, and MAFB.
9. The method of any of embodiments 1-8, wherein the epigenetic loci include cg6522 and POU3F1.
10. The method of any of embodiments 1-9, wherein the tissue sample was obtained with an esophageal brush or sponge.
11. The method of any of embodiments 4-10, wherein the diagnosing with HGD, EAC, or JCA directs an aggressive or experimental treatment for the subject.
12. The method of any of embodiments 4-11, wherein the diagnosing with HGD, EAC, or JCA directs radiofrequency ablation (RFA) and intramucosal resection (EMR).
13. The method of any of embodiments 4-12, wherein the diagnosing with normal or Barrett's esophagus directs monitoring the patient with additional esophageal screenings over time.
14. The method of any of embodiments 4-13, wherein the diagnosing with normal or Barrett's esophagus directs moderate, minimal, or no treatment.
15. The method of any of embodiments 1-14, wherein the detecting includes extracting genomic DNA from cells.
16. The method of embodiment 15, wherein the extracted genomic DNA includes 4 ng of DNA.
17. The method of any of embodiments 1-16, wherein the detecting includes treating extracted genomic DNA with bisulfite.
18. The method of any of embodiments 1-17, wherein the detecting includes performing methylation-specific PCR on bisulfite treated DNA.
19. The method of any of embodiments 1-18, wherein the detecting includes partitioning bisulfite treated DNA into partitions.
20. The method of embodiment 19, wherein the partitions are water-in-oil droplets.
21. The method of embodiments 19 or 20, wherein each partition contains one or zero fragments of bisulfite treated DNA.
22. The method of any of embodiments 19-21, wherein each partition includes an amplification mixture.
23. The method of embodiment 22, wherein the amplification mixture includes at least one primer or primer pair, at least one probe, at least one replication enzyme, and deoxynucleotide and/or nucleotide triphosphates.
24. The method of embodiment 23, wherein the at least one primer pair has the sequences as set forth in SEQ ID NOs: 9 and 10 or sequences having at least 95% sequence identity thereto.
25. The method of embodiment 24, wherein the at least one probe has the sequence as set forth in SEQ ID NO: 11 or a sequence having at least 95% sequence identity thereto.
26 The method of embodiment 23, wherein the at least one primer pair has the sequences as set forth in SEQ ID NOs: 12 and 13 or sequences having at least 95% sequence identity thereto.
27. The method of embodiment 26, wherein the at least one probe has the sequence as set forth in SEQ ID NO: 14 or a sequence having at least 95% sequence identity thereto.
28. The method of embodiment 23, wherein the at least one primer pair has the sequences as set forth in SEQ ID NOs: 15 and 16 or sequences having at least 95% sequence identity thereto.
29. The method of embodiment 28, wherein the at least one probe has the sequence as set forth in SEQ ID NO: 17 or a sequence having at least 95% sequence identity thereto.
30. The method of embodiment 23, wherein the at least one primer pair has the sequences as set forth in SEQ ID NOs: 9, 10, 15, and 16, or sequences having at least 95% sequence identity thereto.
31. The method of embodiment 30, wherein the at least one probe has the sequence as set forth in SEQ ID NOs: 11 and 17 or sequences having at least 95% sequence identity thereto.
32. The method of embodiment 23, wherein the at least one primer pair has the sequences as set forth in SEQ ID NOs: 9, 10, 12, 13, 15, and 16, or sequences having at least 95% sequence identity thereto.
33. The method of embodiment 30, wherein the at least one probe has the sequence as set forth in SEQ ID NOs: 11, 14, and 17 or sequences having at least 95% sequence identity thereto.
34. The method of embodiment 23, further including thermocycling the partitions to perform PCR.
35. The method of embodiment 34, further including analyzing partitions for the presence of PCR-positive partitions.
36. A kit for distinguishing (i) a normal or Barrett's esophagus tissue sample from (ii) a high grade dysplasia (HGD) or esophageal adenocarcinoma (EAC), or junctional adenocarcinoma (JCA) tissue sample, the kit including reagents to detect methylation status of cytosines located in one or more epigenetic loci selected from cg6522, POU3F1, YPEL3, and/or MAFB.
37. The kit of embodiment 36, wherein the reagents detect methylation status of cytosines within (i) cg6522, POU3F1, and YPEL3; or (ii) cg6522, POU3F1, YPEL3, and MAFB.
38. The kit of embodiments 36 or 37, wherein the reagents detect methylation status of cytosines within cg6522 and POU3F1.
39. The kit of any of embodiments 36-38, wherein the kit includes a primer pair having the sequences as set forth in SEQ ID NOs: 9 and 10 or sequences having at least 95% sequence identity thereto.
40. The kit of embodiment 39, wherein the kit includes a probe that has the sequence as set forth in SEQ ID NO: 11 or a sequence having at least 95% sequence identity thereto.
41. The kit of any of embodiments 36-40, wherein the kit includes a primer pair having the sequences as set forth in SEQ ID NOs: 12 and 13 or sequences having at least 95% sequence identity thereto.
42. The kit of embodiment 41, wherein the kit includes a probe that has the sequence as set forth in SEQ ID NO: 14 or a sequence having at least 95% sequence identity thereto.
43. The kit of any of embodiments 36-42, wherein the kit includes a primer pair having the sequences as set forth in SEQ ID NOs: 15 and 16 or sequences having at least 95% sequence identity thereto.
44. The kit of embodiment 43, wherein the kit includes a probe that has the sequence as set forth in SEQ ID NO: 17 or a sequence having at least 95% sequence identity thereto.
45. The kit of any of embodiments 36-44, wherein the kit includes primer pairs having the sequences as set forth in SEQ ID NOs: 9, 10, 15, and 16, or sequences having at least 95% sequence identity thereto.
46. The kit of embodiment 45, wherein the kit includes probes that have the sequence as set forth in SEQ ID NOs: 11 and 17 or sequences having at least 95% sequence identity thereto.
47. The kit of any of embodiments 36-46, wherein the kit includes primer pairs having the sequences as set forth in SEQ ID NOs: 9, 10, 12, 13, 15, and 16, or sequences having at least 95% sequence identity thereto.
48. The kit of embodiment 47, wherein the kit includes probes that have the sequence as set forth in SEQ ID NOs: 11, 14, and 17 or sequences having at least 95% sequence identity thereto.
49. The kit of any of embodiments 36-48, further including DNA-fragmenting enzymes.
50. The kit of any of embodiments 36-49, further including bisulfite.
51. The kit of any of embodiments 36-50, further including a replication enzyme.
52. The kit of embodiment 51, wherein the replication enzyme is a thermostable polymerase.
53. The kit of any of embodiments 36-52, further including and deoxynucleotide and/or nucleotide triphosphates
54. The kit of any of embodiments 36-53, further including a reference level.
55. The kit of embodiment 54, wherein the reference level is derived from a population of subjects with normal or Barrett's esophagus.
56. The kit of embodiment 55, wherein an increased methylation value as compared to the reference level distinguishes the sample as HGD, EAC, or JCA.
57. The kit of any of embodiments 36-56, further including a detectable label.
58. The kit of embodiment 57, wherein the detectable label is a radioactive isotope, enzyme, dye, magnetic bead, or biotin.
59. The kit of embodiment 58, wherein the dye is a fluorescent dye.
60. A method of surveilling a Barrett's esophagus patient for the development of HGD, EAC, or JCA including repeatedly
   obtaining a tissue sample derived from the patient;
   detecting a methylation status of cytosines located in an epigenetic loci selected from cg6522, POU3F1, YPEL3, and/or MAFB from the tissue sample to generate a methylation value;
   and
   diagnosing the subject as having (i) Barrett's esophagus or (ii) HGD, EAC, or JCA based on the generated methylation score.
61. The method of embodiment 60, wherein the surveilling includes repeating the steps of embodiment 60 every 3 months to 3 years depending on each patient's condition.
62. The method of embodiments 60 or 61, further including treating the patient for HGD, EAC, or JCA when the subject is diagnosed with HGD, EAC, or JCA.
63. The method of embodiment 62, wherein the treating includes radiofrequency ablation (RFA) and intramucosal resection (EMR).

### (viii) Experimental Example. Results. Discovery of DNA Methylation-Based Biomarkers for Detection of Esophageal High-Grade Dysplastic and Malignant Lesions

A genome-wide DNA methylation profile was conducted using Illumina HumanMethylation450 (HM450) Beadchip arrays on a set of DNA samples from normal esophageal squamous epithelium (SQ) (N=53), non-dysplastic BE (N=71), HGD (N=20), and EAC (N=23). After data normalization, filtering and analyses (as described Yu et al., Subtypes of Barrett's oesophagus and oesophageal adenocarcinoma based on genome-wide methylation analysis. Gut 2018; Jammula et al., Identification of Subtypes of Barrett's Esophagus and Esophageal Adenocarcinoma Based on DNA Methylation Profiles and Integration of Transcriptome and Genome Data. Gastroenterology 2020; Kuester et al., Cancer Lett 2009; 275:117-26; Yu et al., Cancer Epidemiol Biomarkers Prev 2015; 24:1890-7), there were remaining 426,464 CpGs for evaluation. DMP/DMR analyses were performed using limma & minfi bumphunter (See Methods). Twenty-four CpGs were identified to be significantly hypermethylated in the HGD and EAC samples compared to the NDBE or normal SQ samples, using the following cutoff values: difference in mean beta-value >0.15 (HGD and EAC vs. BE and SQ), baseline beta values in SQ or NDBE<0.25, and false discovery rate q<0.001. These 24 top candidate CpGs were selected for further assessment and validation.

The association of HGD, EAC, SQ or BE status with potential confounding factors, such as age and gender was assessed. Age or gender were not found to be significant confounding variables (ANOVA p=0.1 for age, Chi-Square test p=0.5 for gender). Race was not assessed as a confounding variate because the majority of subjects from which samples were obtained were Caucasians (Table 2).

TABLE 2
Patient Demographic Information

| | | Biopsy test | | |
|---|---|---|---|---|
| | HM450 assay | set for MS- | Brushing | Brushing |
| Subject | Discovery | ddPCR | training | validation |
| Information | set | assays | set | set |
| Total | 167 | 76 | 194 | 129 |
| Gender | | | | |
| Female | 32 | 10 | 54 | 28 |
| Male | 135 | 66 | 140 | 101 |
| Age | | | | |
| Range | 21-93 (64) | 46-82 (64) | 21-92 (64.2) | 28-93 (64.3) |
| (Mean) | | | | |
| Smoking | | | | |
| Yes | 36 | 47 | 114 | 57 |
| (current or | | | | |
| former) | | | | |
| Never | 15 | 25 | 72 | 64 |
| NA | 116 | 4 | 8 | 8 |

There is no significant difference between HGD/EAC vs SQ/BE for any variable in the three data sets (p>0.05). Chi-Square test for category variables, ANOVA F-test for numerical variables.

Assessment of top candidate methylated DNA biomarkers in an independent training set of endoscopic biopsy samples. Ultra-sensitive highly-precise droplet digital PCR assays that measure the methylation level of the 150 bp region surrounding the target CpGs on the HM450 arrays for the top candidate CpGs were developed. The limit of detection and limit of quantification for each assay was determined. The best performing assays (for cg6522, YPEL3, POU3F1, MAFB) were then selected and the assays were used on DNA extracted from clinical samples. The primer and probe sequences for these MS-ddPCR assays are listed in Table 1.

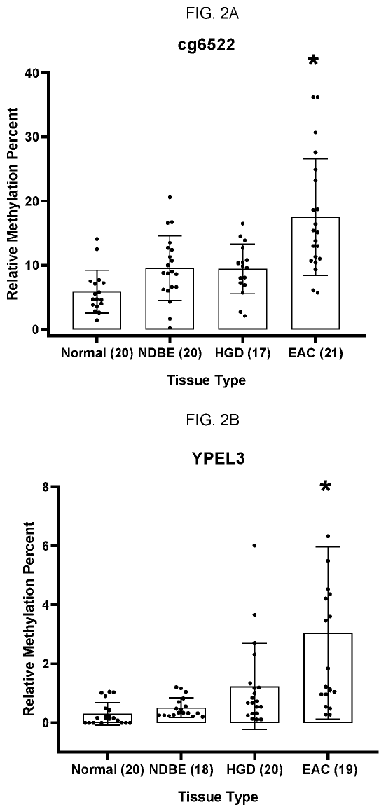

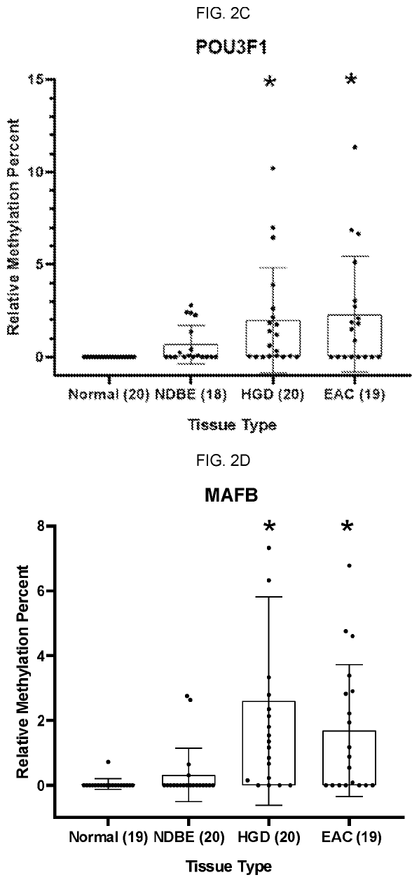

Next, the MS-ddPCR assays for cg6522, YPEL3, POU3F1, and MAFB were used to assess their methylation status in an independent DNA samples extracted from endoscopic esophageal biopsies (Training set: SQ=20, NDBE=18, HGD=20, EAC=19). As shown in FIGS. 2A-2D, the mean methylation levels of all four genes were significantly elevated in EAC, compared to the SQ or NDBE (*, p<0.01 vs. SQ or NDBE). The median methylation level of POU3F1 and MAFB was significantly elevated in both HGD and EAC samples (*, p<0.01 vs. SQ or NDBE).

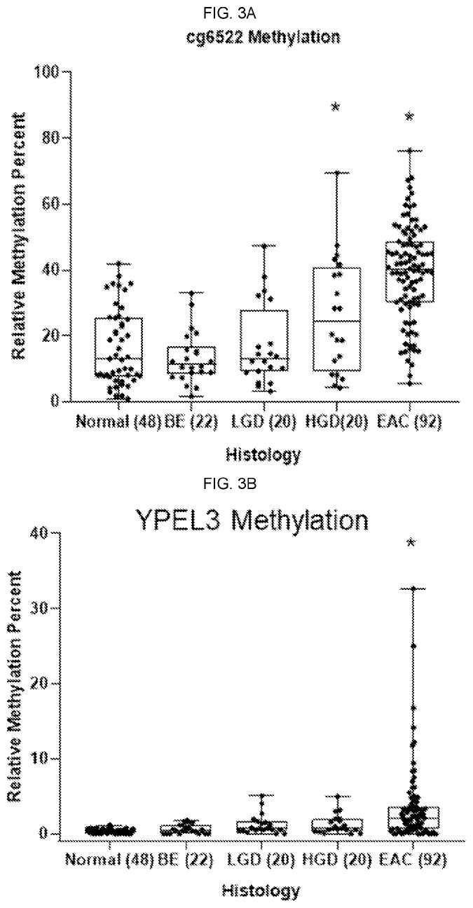

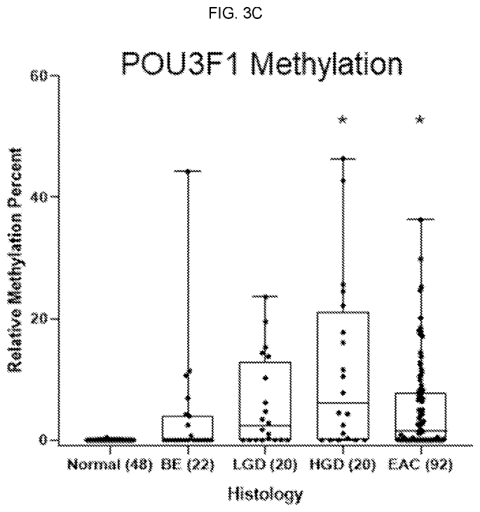

Assessment of top candidate methylated DNA biomarkers in an independent set of esophageal samples. To determine the accuracy of the methylation thresholds for the MS-ddPCR assays for cg6522, YPEL3, and POU3F1 identified in the training set of samples, an independent set of esophageal cytology brushings from 181 individuals composed of 51 controls with normal esophageal endoscopic exams, 20 with NDBE, 22 with HGD and 88 cases with EAC (termed Validation set) was examined. There is no statistically significant difference in the gender or age of the subjects between the training and validation sets. As shown in FIGS. 3A-3C, the normal SQ and non-dysplastic BE samples showed low level of methylation of cg6522, YPEL3, and POU3F1, whereas DNA from HGD and EAC samples had significantly higher methylation levels (p<0.01, respectively).

Development of HGD and EAC early detection biomarker panel. The performance of the MS-ddPCR assays for cg6522, YPEL3, and POU3F1 as individual biomarkers and as panels of multiple markers for detecting HGD and/or EAC was next determined. the receiver operating characteristic (ROC) curve for the candidate biomarker assays was determined using logistic regression model and area under the curve (AUC) for each individual marker and any combination of cg6522, YPEL3, and POU3F1. The model based on a 2-marker panel composed of cg6522 and POU3F1, which was logit(p)=−3.75+0.1130×cg6522+0.1926×POU3F1, was selected as the best model for detecting HGD/EAC. When the cutoff p value was set at 0.45, the AUC in the brushing sample set 1 was 0.93 with a sensitivity of detecting HGD and EAC at 80% and 87.2% respectively, and at specificity for normal controls and NDBE at 85.4% and 94.7%, respectively (Table 3).

TABLE 3
Performance of disclosed markers in
training set of Esophageal brushings

| Common Dataset for All Markers | | | | | |
|---|---|---|---|---|---|
| | # of | | | | Model 1 |
| | samples | Cg6522 | YPEL3 | POU3F1 | Prob_HGD_EAC |
| Cutoff for | | 40 | 2.5 | 12 | 0.45 |
| Positivity | | | | | |
| Specificity | 48 | 97.9% | 100.0% | 100.0% | 85.4% |
| N's | | | | | |
| Specificity | 19 | 100.0% | 100.0% | 100.0% | |
| BE | | | | | |
| Sens LGD | 20 | 5.0% | 15.0% | 25.0% | 45.0% |
| Sens HGD | 20 | 25.0% | 15.0% | 35.0% | 80.0% |
| Sens EAC | 87 | 50.6% | 43.7% | 16.1% | 87.4% |

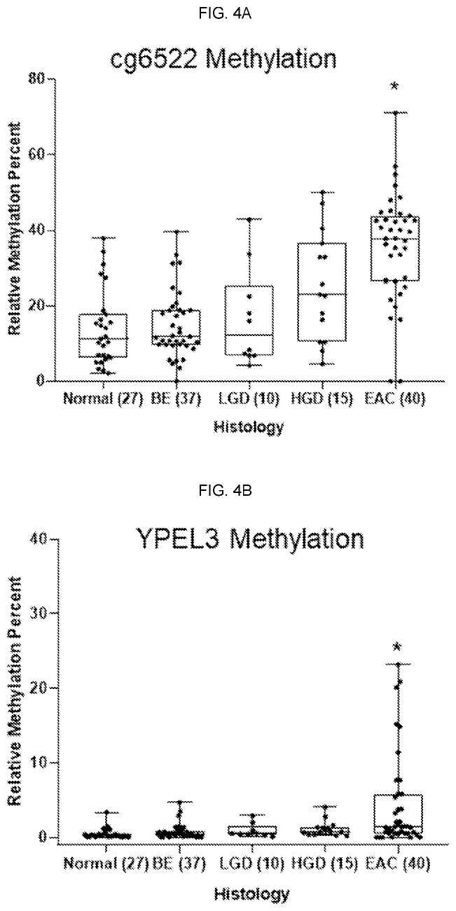

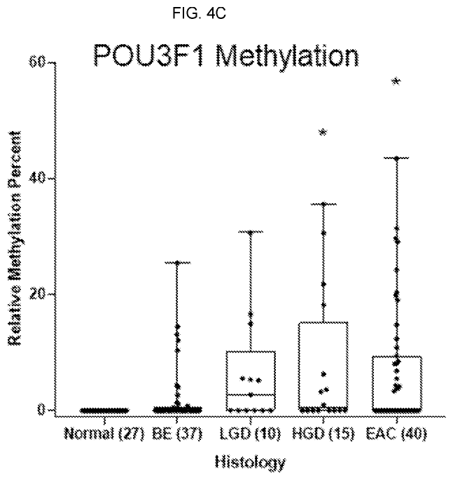

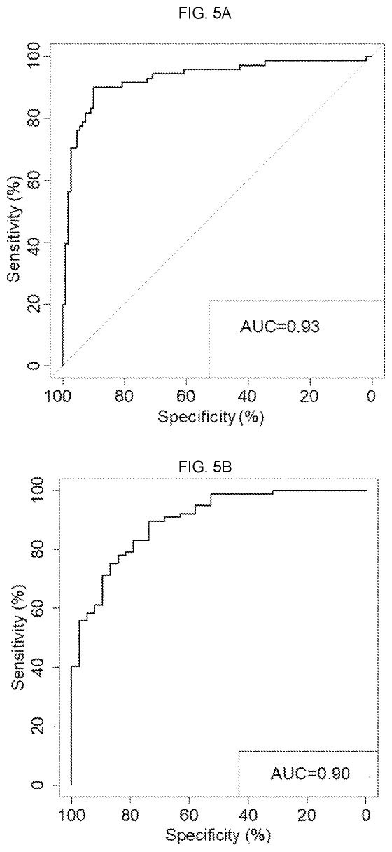

Assessment of models in an independent set of bushing samples. To validate the performance of the model, the methylation levels of individual markers cg6522, YPEL3, POU3F1 were examined in a second independent set of esophageal cytology brushings from 27 control patients with normal esophageal endoscopic exams, 37 with NDBE, 10 with LGD, 15 with HGD and 40 cases with EAC (FIGS. 4A-4C). This model displays a statistically significant high specificity for normal squamous tissue and NDBE at 92.6% and 83.8%, with a sensitivity of 67% and 82.5% for detection of HGD and EAC, respectively (Table 4).

TABLE 4
Performance of disclosed markers in Validation
set of Esophageal brushings

| Common Dataset for All Markers | | | | |
|---|---|---|---|---|
| | # samples | Cg6522 | YPEL3 | Prob_HGD EAC |
| Cutoff | | 40 | 2.5 | 0.45 |
| Specificity | 27 | 100.0% | 96.3% | 92.6% |
| N's | | | | |
| Specificity | 37 | 100.0% | 91.9% | 83.8% |
| BE | | | | |
| Sens LGD | 10 | 10% | 10% | 40% |
| Sens HGD | 15 | 20% | 13% | 67% |
| Sens EAC | 40 | 45.0% | 35.0% | 82.5% |

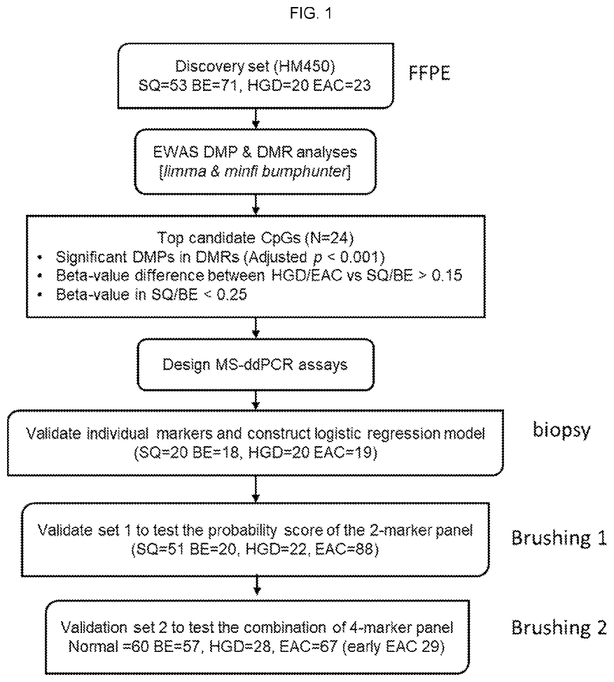

Materials and Methods. Study design. The overall study was a non-randomized observational study. Study size was not pre-specified. The primary endpoint of detection of Barrett's esophagus and related progressed lesions was pre-specified before study initiation. All laboratory samples were assayed by investigators blinded to the clinical status of the subjects from whom the brushing samples were obtained (FIG. 1).

Tissue Samples. The discovery set of formalin-fixed paraffin-embedded (FFPE) slides was obtained from Case Western Reserve University/University Hospitals of Cleveland (Cleveland, OH), the University of Michigan Medical School (Ann Arbor, MI), and University of North Carolina School of Medicine (Chapel Hill, NC), following protocols approved by the Institutional Review Board of each institution. H&E-stained slides were created for each sample and examined by an expert pathologist to confirm diagnosis and identify precise areas with the histological subtypes of interest. Unstained slides were then matched with the annotated H&E slides and a sterile razor blade was used to remove the tissue for DNA extraction. In cases with mixed histology, special care was taken to separate histological subtypes before extraction. The comprehensive discovery set included a total of 53 normal, 71 BE, 20 HGD, and 23 EAC samples run on Illumina HumanMethylation450 BeadChip arrays (HM450 arrays).

The training set consisted of an independent set of endoscopic biopsy and endomucosal resection (EMR) samples obtained from University of Washington Medical Center (UWMC), with a total of 20 normal, 18 non-dysplastic Barrett's (NDBE), 20 HGD, and 19 EAC samples. DNA from normal samples was extracted from fresh-frozen tissue and all other tissue types were extracted from FFPE slides examined by an experienced pathologist.

To validate findings from esophageal biopsies of methylated biomarkers for detection of HGD/EAC, two independent sets of endoscopic cytology brushings were collected from Case Western Reserve University/University Hospitals of Cleveland (Cleveland, OH) as reported in Moinova et al., Sci Transl Med 2018; 10; Kadri et al., BMJ; 341:c4372. The first validation set of samples consisted of 181 endoscopic brushings (51 Normal, 20 BE, 22 HGD, and 88 EAC samples) and the 2nd consisted of 212 endoscopic brushings (60 Normal, 57 BE, 28 HGD, and 67 EAC samples). The patients had a previously confirmed diagnosis by endoscopic biopsy. All brushing specimens were obtained at the time of the endoscopic exam using a through-the-scope cytology brush, prior to passage of the endoscope through the area of interest. Demographic information on the subjects in each sample set can be found in Table 1. Control subjects had no endoscopic evidence of BE and no histological evidence of intestinal metaplasia if a clinical biopsy was obtained from either the distal esophagus or gastroesophageal junction.

Bisulfite DNA preparation. Genomic DNA was extracted from fresh-frozen tissues with the DNeasy Blood and Tissue Kit, following manufacturer's instructions and eluted into a total volume of 100 μL. The QIAamp DNA FFPE Tissue Kit (Qiagen) was used to extract genomic DNA from FFPE tissues according to manufacturer's instructions with the modification of lysing all tissues overnight. Samples were then eluted into 25-100 uL, dependent upon tissue size. Quant-iT PicoGreen DNA assay kit (Life Technologies) was used to quantify genomic DNA before bisulfite conversion. The EZ DNA Methylation Kit (ZymoResearch) was used for bisulfite conversion. All samples were eluted into 20 uL final volume (10 ul for Arrays, 20 ul for ddPCR).

HM450 arrays and genome-wide differential methylation analysis. Infinium HD FFPE DNA Restore Kit (Illumina Inc.) was used to process bisulfite-converted DNA according to manufacturer's instructions. DNA samples were submitted to the Genomics Core at the Fred Hutchinson Cancer Research Center (FHCRC) for processing and subsequently run on the HM450 arrays according to manufacturer's instructions (Illumina Inc.). Data acquisition, normalization, filtering, and analysis were conducted as described in Yu et al., Subtypes of Barrett's oesophagus and oesophageal adenocarcinoma based on genome-wide methylation analysis. Gut 2018 and Yu et al., Cancer Epidemiol Biomarkers Prev 2015; 24:1890-7. Each CpG site was evaluated for differential methylation between Normal/BE and HGD/EAC by comparing mean ‘beta-values’ for the two groups (0.0=0% methylation, 1.0=100% methylation) using R Limma with adjustment for the sample age and gender. Differentially methylated genomic regions were also analyzed between the two groups using R minfi bumphunter (Aryee et al. Minfi: a flexible and comprehensive Bioconductor package for the analysis of Infinium DNA methylation microarrays. Bioinformatics 2014; 30:1363-9). CpG sites with statistically significant higher methylation in HGD/EAC vs. Normal/BE with the adjusted p value <0.001, beta-value difference >0.1, baseline beta-value <0.2 and located in differentially methylated regions were considered for assay development.

Methylation-specific ddPCR. MethyLight PCR assays were designed for four CpGs, referred to as cg6522, YPEL3, POU3F1, and MAFB (cg4156522, cg16348385, cg38512601, and cg18251612, respectively) and methylation-specific ddPCR was run on esophageal biopsy and brushing DNA samples. ABI Primer Express Software version 3.0.1 primer/probe test tool was used to manually design the primer and probe sequences for each region. The C-LESS-C1 assay was used as a methylation-independent control as previously described (Weisenberger, et al., Nucleic Acids Res 2008; 36:4689-98; Yu, et al., Methods Mol Biol 2018; 1768:363-383; Yu et al., Epigenetics 2015; 10:803-9). A list of all primer/probe sequences used can be found in Table 2.

Methylation-specific ddPCR reactions contained 2×ddPCR Supermix for Probes (no dUTP) (BioRad), locus-specific primers (900 nmol/L), and locus-specific probes (250 nmol/L). Each reaction was done in duplex with the CpG of interest and the control assay, as previously described (Yu, Heinzerling, & Grady, Methods Mol Biol 2018; 1768:363-383; Yu et al., Epigenetics 2015; 10:803-9). Bisulfite-converted samples were used as template DNA, 100% methylated EpiTect Methyl DNA was used as positive control, and 100% unmethylated EpiTect Unmethyl DNA (Qiagen) was used as negative control; all samples were run in duplicate. Reactions underwent droplet generation using the QX200 droplet generator (BioRad). Thermocycler conditions were performed in the T100 Thermal Cycler (BioRad): 95° C. for 10 min, 45 cycles of 94° C. for 30 seconds followed by 60° C. for 1 min, 98° C. for 10 min, and hold at 4° C. indefinitely. Results were generated using the QX200 Droplet Reader (BioRad) and data was analyzed with QuantaSoft Software, Regulatory Edition as previously described in Yu, Heinzerling, & Grady, Methods Mol Biol 2018; 1768:363-383; and Yu et al., Epigenetics 2015; 10:803-9.

Methylation status in both validation sets was reported as relative methylation percentage (RM %), calculated as a ratio percentage of the amount of target methylated alleles (cg6522, YPEL3, POU3F1, or MAFB) over total DNA measured by the C-LESS-C1 assay.

Classification/prediction for HGD/EAC. A logistic regression model was trained on the biopsy sample set (training set) as logit(p)=log(p/(1−p)=β₀+β₁x₁+ . . . +βₘxₙ, where p and β stand for the probability score of the specified sample being positive and the intercept or coefficient of each marker. The trained model was then tested on the brushing samples set (validation set). Different combinations of the candidate markers were compared based on area under the ROC curve (AUC), with the largest AUC indicating the best performance. The diagnostic sensitivity, specificity and accuracy, defined as (true positives+true negatives)/total sample size, were calculated for the best model in both of the training and testing sets.

Statistical Analysis. Differential methylation analysis of the discovery HM450 array data was performed using R minfi and limma. Results shown here from the training and validation sample sets comparing methylation levels between different histologic groups were generated using GraphPad Prism version 7 (GraphPad Software Inc.). Chi-Square test and ANOVA were used to test difference of category and numeric clinical variables between different groups.
(ix) Closing Paragraphs. The disclosure is not limited to the particularly referenced gene and associated nucleic acid and protein sequences but instead also encompasses sequences including 80% sequence identity; 81% sequence identity; 82% sequence identity; 83% sequence identity; 84% sequence identity; 85% sequence identity; 86% sequence identity; 87% sequence identity; 88% sequence identity; 89% sequence identity; 90% sequence identity; 91% sequence identity; 92% sequence identity; 93% sequence identity; 94% sequence identity; 95% sequence identity; 96% sequence identity; 97% sequence identity; 98% sequence identity or 99% sequence identity to a gene sequence referenced herein.

“% sequence identity” refers to a relationship between two or more sequences, as determined by comparing the sequences. In the art, “identity” also means the degree of sequence relatedness between sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part|(Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine sequence identity are designed to give the best match between the sequences tested. Methods to determine sequence identity and similarity can be found in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. Within the context of this disclosure, it will be understood that where sequence analysis software is used for analysis, the results of the analysis are based on the “default values” of the program referenced. “Default values” mean any set of values or parameters which originally load with the software when first initialized.

Moreover, the term “gene” can include not only coding sequences but also regulatory regions such as promoters, enhancers, and termination regions. The term further can include all introns and other DNA sequences spliced from the mRNA transcript, along with variants resulting from alternative splice sites. Portions of complete gene sequences can be referenced as is understood by one of ordinary skill in the art.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” As used herein, the transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. As used herein, a material effect would cause a statistically-significant reduction in ability to normal and Barrett's esophagus from HGD, EAC, or JCA.

In the context of nucleotide sequences, “reagents to detect”, “target specific probes” and “specific for” mean that the nucleotide sequences interact with target sequences (or sequences related to the target sequence based on the particular assay) with sufficient specificity and strength to reliably detect methylation status of cytosines within the targeted sequence. Particular nucleotide sequences with these characteristics can be readily generated and identified by those of ordinary skill in the art, based on the teachings of the current disclosure and with reference to numerous publicly available resources and databases.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that can vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. It is anticipated that one or more members of a group can be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that can be employed are within the scope of the invention. Thus, by way of example, but not of limitation, alternative configurations of the present invention can be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention can be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3^{rd} Edition or a dictionary known to those of ordinary skill in the art, such as the Oxford Dictionary of Biochemistry and Molecular Biology (Ed. Anthony Smith, Oxford University Press, Oxford, 2004).

What is claimed is:

1. A method to test a subject for the presence of high grade dysplasia (HGD), esophageal adenocarcinoma (EAC), or junctional adenocarcinoma (JCA) in the subject, the method comprising:
obtaining a tissue sample derived from the subject;
detecting a methylation status of cytosines located in epigenetic loci consisting of cg6522 and POU3F1 from the tissue sample to generate a methylation value;
and
determining that high grade dysplasia (HGD), esophageal adenocarcinoma (EAC), or junctional adenocarcinoma (JCA) is present or absent in the subject based on the generated methylation value.

2. A method to test a subject for the presence of high grade dysplasia (HGD), esophageal adenocarcinoma (EAC), or junctional adenocarcinoma (JCA) in the subject, the method comprising:
obtaining a tissue sample derived from the subject;
detecting a methylation status of cytosines located in epigenetic loci consisting of cg6522, POU3F1, YPEL3, and MAFB from the tissue sample to generate a methylation value;
and
determining that high grade dysplasia (HGD), esophageal adenocarcinoma (EAC), or junctional adenocarcinoma (JCA) is present or absent in the subject based on the generated methylation value.

3. A method to test a subject for the presence of high grade dysplasia (HGD), esophageal adenocarcinoma (EAC), or junctional adenocarcinoma (JCA) in the subject, the method comprising:
obtaining a tissue sample derived from the subject;
detecting a methylation status of cytosines located in an epigenetic loci selected from cg6522, POU3F1, YPEL3, and/or MAFB from the tissue sample to generate a methylation value;
and
determining that high grade dysplasia (HGD), esophageal adenocarcinoma (EAC), or junctional adenocarcinoma (JCA) is present or absent in the subject based on the generated methylation value.

4. The method of claim 3, wherein the generated methylation value is increased in comparison to a reference level; the HGD, EAC, or JCA is present; and the subject is diagnosed with the HGD, EAC, or JCA.

5. The method of claim 3, wherein the generated methylation value is not increased in comparison to a reference level and the HGD, EAC, or JCA is not present.

6. A method to diagnose a subject as having (i) a normal or Barrett's esophagus tissue sample or (ii) a high grade dysplasia (HGD), esophageal adenocarcinoma (EAC), or junctional adenocarcinoma (JCA) tissue sample, the method comprising:
obtaining a tissue sample derived from the subject;
detecting a methylation status of cytosines located in an epigenetic loci selected from cg6522, POU3F1, YPEL3, and/or MAFB from the tissue sample to generate a methylation value;
and
diagnosing the subject as having (i) normal or Barrett's esophagus or (ii) HGD, EAC, or JCA based on the generated methylation score.

7. The method of claim 6, wherein the subject is diagnosed as having normal or Barrett's esophagus when the generated methylation value is not increased over a reference level.

8. The method of claim 6, wherein the subject is diagnosed as having HGD, EAC, or JCA when the generated methylation value is increased over a reference level.

9. The method of claim 4, 5, 7, or 8, wherein the reference level is derived from a population of subjects having normal or Barrett's esophagus.

10. The method of claim 3 or 6, wherein the epigenetic loci comprise or consist of (i) cg6522, POU3F1, and YPEL3; or (ii) cg6522, POU3F1, YPEL3, and MAFB.

11. The method of claim 3 or 6, wherein the epigenetic loci comprise or consist of cg6522 and POU3F1.

12. The method of claim 3 or 6, wherein the tissue sample was obtained with an esophageal brush or sponge.

13. The method of claim 4 or 8, wherein the diagnosing with HGD, EAC, or JCA directs an aggressive or experimental treatment for the subject.

14. The method of claim 4 or 8, wherein the diagnosing with HGD, EAC, or JCA directs radiofrequency ablation (RFA) and intramucosal resection (EMR).

15. The method of claim 7, wherein the diagnosing with normal or Barrett's esophagus directs monitoring the patient with additional esophageal screenings over time.

16. The method of claim 7, wherein the diagnosing with normal or Barrett's esophagus directs moderate, minimal, or no treatment.

17. The method of claim 3 or 6, wherein the detecting comprises extracting genomic DNA from cells.

18. The method of claim 17, wherein the extracted genomic DNA comprises 4 ng of DNA.

19. The method of claim 3 or 6, wherein the detecting comprises treating extracted genomic DNA with bisulfite.

20. The method of claim 3 or 6, wherein the detecting comprises performing methylation-specific PCR on bisulfite treated DNA.

21. The method of claim 3 or 6, wherein the detecting comprises partitioning bisulfite treated DNA into partitions.

22. The method of claim 21, wherein the partitions are water-in-oil droplets.

23. The method of claim 21, wherein each partition contains one or zero fragments of bisulfite treated DNA.

24. The method of claim 21, wherein each partition comprises an amplification mixture.

25. The method of claim 24, wherein the amplification mixture comprises at least one primer or primer pair, at least one probe, at least one replication enzyme, and deoxynucleotide and/or nucleotide triphosphates.

26. The method of claim 25, wherein the at least one primer pair has the sequences as set forth in SEQ ID NOs: 9 and 10 or sequences having at least 95% sequence identity thereto.

27. The method of claim 26, wherein the at least one probe has the sequence as set forth in SEQ ID NO: 11 or a sequence having at least 95% sequence identity thereto.

28. The method of claim 25, wherein the at least one primer pair has the sequences as set forth in SEQ ID NOs: 12 and 13 or sequences having at least 95% sequence identity thereto.

29. The method of claim 28, wherein the at least one probe has the sequence as set forth in SEQ ID NO: 14 or a sequence having at least 95% sequence identity thereto.

30. The method of claim 25, wherein the at least one primer pair has the sequences as set forth in SEQ ID NOs: 15 and 16 or sequences having at least 95% sequence identity thereto.

31. The method of claim 30, wherein the at least one probe has the sequence as set forth in SEQ ID NO: 17 or a sequence having at least 95% sequence identity thereto.

32. The method of claim 25, wherein the at least one primer pair has the sequences as set forth in SEQ ID NOs: 9, 10, 15, and 16, or sequences having at least 95% sequence identity thereto.

33. The method of claim 32, wherein the at least one probe has the sequence as set forth in SEQ ID NOs: 11 and 17 or sequences having at least 95% sequence identity thereto.

34. The method of claim 25, wherein the at least one primer pair has the sequences as set forth in SEQ ID NOs: 9, 10, 12, 13, 15, and 16, or sequences having at least 95% sequence identity thereto.

35. The method of claim 32, wherein the at least one probe has the sequence as set forth in SEQ ID NOs: 11, 14, and 17 or sequences having at least 95% sequence identity thereto.

36. The method of claim 25, further comprising thermocycling the partitions to perform PCR.

37. The method of claim 36, further comprising analyzing partitions for the presence of PCR-positive partitions.

38. A kit for distinguishing (i) a normal or Barrett's esophagus tissue sample from (ii) a high grade dysplasia (HGD) or esophageal adenocarcinoma (EAC), or junctional adenocarcinoma (JCA) tissue sample, the kit comprising reagents to detect methylation status of cytosines located in one or more epigenetic loci selected from cg6522, POU3F1, YPEL3, and/or MAFB.

39. The kit of claim 38, wherein the reagents detect methylation status of cytosines within (i) cg6522, POU3F1, and YPEL3; or (ii) cg6522, POU3F1, YPEL3, and MAFB.

40. The kit of claim 38, wherein the reagents detect methylation status of cytosines within cg6522 and POU3F1.

41. The kit of claim 38, wherein the kit comprises a primer pair having the sequences as set forth in SEQ ID NOs: 9 and 10 or sequences having at least 95% sequence identity thereto.

42. The kit of claim 41, wherein the kit comprises a probe that has the sequence as set forth in SEQ ID NO: 11 or a sequence having at least 95% sequence identity thereto.

43. The kit of claim 38, wherein the kit comprises a primer pair having the sequences as set forth in SEQ ID NOs: 12 and 13 or sequences having at least 95% sequence identity thereto.

44. The kit of claim 43, wherein the kit comprises a probe that has the sequence as set forth in SEQ ID NO: 14 or a sequence having at least 95% sequence identity thereto.

45. The kit of claim 38, wherein the kit comprises a primer pair having the sequences as set forth in SEQ ID NOs: 15 and 16 or sequences having at least 95% sequence identity thereto.

46. The kit of claim 45, wherein the kit comprises a probe that has the sequence as set forth in SEQ ID NO: 17 or a sequence having at least 95% sequence identity thereto.

47. The kit of claim 38, wherein the kit comprises primer pairs having the sequences as set forth in SEQ ID NOs: 9, 10, 15, and 16, or sequences having at least 95% sequence identity thereto.

48. The kit of claim 47, wherein the kit comprises probes that have the sequence as set forth in SEQ ID NOs: 11 and 17 or sequences having at least 95% sequence identity thereto.

49. The kit of claim 38, wherein the kit comprises primer pairs having the sequences as set forth in SEQ ID NOs: 9, 10, 12, 13, 15, and 16, or sequences having at least 95% sequence identity thereto.

50. The kit of claim 49, wherein the kit comprises probes that have the sequence as set forth in SEQ ID NOs: 11, 14, and 17 or sequences having at least 95% sequence identity thereto.

51. The kit of claim 38, further comprising DNA-fragmenting enzymes.

52. The kit of claim 38, further comprising bisulfite.

53. The kit of claim 38, further comprising a replication enzyme.

54. The kit of claim 53, wherein the replication enzyme is a thermostable polymerase.

55. The kit of claim 38, further comprising and deoxynucleotide and/or nucleotide triphosphates

56. The kit of claim 38, further comprising a reference level.

57. The kit of claim 56, wherein the reference level is derived from a population of subjects with normal or Barrett's esophagus.

58. The kit of claim 57, wherein an increased methylation value as compared to the reference level distinguishes the sample as HGD, EAC, or JCA.

59. The kit of claim 38, further comprising a detectable label.

60. The kit of claim 59, wherein the detectable label is a radioactive isotope, enzyme, dye, magnetic bead, or biotin.

61. The kit of claim 60, wherein the dye is a fluorescent dye.

62. A method of surveilling a Barrett's esophagus patient for the development of HGD, EAC, or JCA comprising repeatedly
obtaining a tissue sample derived from the patient;
detecting a methylation status of cytosines located in an epigenetic loci selected from cg6522, POU3F1, YPEL3, and/or MAFB from the tissue sample to generate a methylation value;
and
diagnosing the subject as having (i) Barrett's esophagus or (ii) HGD, EAC, or JCA based on the generated methylation score.

63. The method of claim 62, wherein the surveilling includes repeating the steps of claim 60 every 3 months to 3 years depending on each patient's condition.

64. The method of claim 62, further comprising treating the patient for HGD, EAC, or JCA when the subject is diagnosed with HGD, EAC, or JCA.

65. The method of claim 64, wherein the treating comprises radiofrequency ablation (RFA) and intramucosal resection (EMR).


SEQUENCE LISTING (20 sequences)

SEQ ID NO: 1 - DNA, 156 bp, Homo sapiens
  source 1..156: mol_type = genomic DNA
aatgcagtcggccctgcgcatgtggtgtctgagcagggcccgagaccttccagggaggggagaaatactttttggctgactctcctgactctgtgtttcctacaattctacaacaaagatccaggaacatgagaaaaggaaaatgagaaaatgagt

SEQ ID NO: 2 - DNA, 97 bp, Homo sapiens
  source 1..97: mol_type = genomic DNA
cattggctgcccgcggagctgcctcccgcctcccctgcccggcccccgccccgcgcgcccgcccgccaccctgcccggccgcccagagctctccatt

SEQ ID NO: 3 - DNA, 147 bp, Homo sapiens
  source 1..147: mol_type = genomic DNA
agggagtaggtgggctgtcaggacctgggccacacacatgcgaggcactcccagagccgtggggactcgctctgtcacactgggctgctctctcctttccccagagccagcagcctctccggggaccagaggcgtctcggttttgac

SEQ ID NO: 4 - DNA, 149 bp, Homo sapiens
  source 1..149: mol_type = genomic DNA
cgctcgcagccgctcgcagctcggcggtgcagctgtgctggatccggcggcgccgcagccttttatcgcctcctgatgtcactggggtgcgggggcccgggcggcccggtgcgcgggccaatagctgcacggcctccgcggcccagcgg

SEQ ID NO: 5 - DNA, 156 bp, Homo sapiens
  source 1..156: mol_type = genomic DNA
aatgtagtcggttttgcgtatgtggtgtttgagtagggttcgagattttttagggaggggagaaatattttttggttgatttttttgattttgtgttttttataattttataataaagatttaggaatatgagaaaaggaaaatgagaaaatgagt

SEQ ID NO: 6 - DNA, 97 bp, Homo sapiens
  source 1..97: mol_type = genomic DNA
tattggttgttcgcggagttgtttttcgttttttttgttcggttttcgtttcgcgcgttcgttcgttattttgttcggtcgtttagagttttttatt

SEQ ID NO: 7 - DNA, 147 bp, Homo sapiens
  source 1..147: mol_type = genomic DNA
agggagtaggtgggttgttaggatttgggttatatatatgcgaggtatttttagagtcgtggggattcgttttgttatattgggttgtttttttttttttttagagttagtagttttttcggggattagaggcgtttcggttttgat

SEQ ID NO: 8 - DNA, 149 bp, Homo sapiens
  source 1..149: mol_type = genomic DNA
ccgctaaaccgcgaaaaccgtacaactattaacccgcgcaccgaaccgcccgaacccccgcaccccaataacatcaaaaaacgataaaaaactacgacgccgccgaatccaacacaactacaccgccgaactacgaacgactacgaacg

SEQ ID NO: 9 - DNA, 24 bp, synthetic construct
  source 1..24: mol_type = other DNA
aatgtagtcggttttgcgtatgtg

SEQ ID NO: 10 - DNA, 27 bp, synthetic construct
  source 1..27: mol_type = other DNA
actcattttctcattttccttttctca

SEQ ID NO: 11 - DNA, 17 bp, synthetic construct
  source 1..17: mol_type = other DNA
  misc_feature 1: note = FAM dye in 5' terminus of probe
  misc_feature 17: note = Minor Groove Binding Nonfluorescent Quencher in 3' terminus of probe
agtagggttcgagattt

SEQ ID NO: 12 - DNA, 24 bp, synthetic construct
  source 1..24: mol_type = other DNA
agggagtaggtgggttgttaggat

SEQ ID NO: 13 - DNA, 23 bp, synthetic construct
  source 1..23: mol_type = other DNA
atcaaaaccgaaacgcctctaat

SEQ ID NO: 14 - DNA, 17 bp, synthetic construct
  source 1..17: mol_type = other DNA
  misc_feature 1: note = FAM dye in 5' terminus of probe
  misc_feature 17: note = Minor Groove Binding Nonfluorescent Quencher in the 3' terminus of probe
tagagtcgtggggattc

SEQ ID NO: 15 - DNA, 21 bp, synthetic construct
  source 1..21: mol_type = other DNA
tattggttgttcgcggagttg

SEQ ID NO: 16 - DNA, 27 bp, synthetic construct
  source 1..27: mol_type = other DNA
aataaaaaactctaaacgaccgaacaa

SEQ ID NO: 17 - DNA, 15 bp, synthetic construct
  source 1..15: mol_type = other DNA
  misc_feature 1: note = FAM dye in 5' terminus of probe
  misc_feature 15: note = Minor Groove Binding Nonfluorescent Quencher in the 3' terminus of probe
tttcgcgcgttcgtt

SEQ ID NO: 18 - DNA, 18 bp, synthetic construct
  source 1..18: mol_type = other DNA
ccgctaaaccgcgaaaac

SEQ ID NO: 19 - DNA, 23 bp, synthetic construct
  source 1..23: mol_type = other DNA
cgttcgtagtcgttcgtagttcg

SEQ ID NO: 20 - DNA, 15 bp, synthetic construct
  source 1..15: mol_type = other DNA
  misc_feature 1: note = FAM dye in 5' terminus of probe
  misc_feature 15: note = Minor Groove Binding Nonfluorescent Quencher in the 3' terminus of probe
actacgacgccgccg